(19) United States
(12) Patent Application Publication
(10) Pub. No.: US 2023/0073888 A1
(43) Pub. Date: Mar. 9, 2023

(54) TREATMENT OF ATOPIC DERMATITIS WITH ANTI-TSLP ANTIBODY

(71) Applicant: AMGEN INC., Thousand Oaks, CA (US)

(72) Inventors: Jane R. Parnes, Agoura Hills, CA (US); Steven Komjathy, Thousand Oaks (CA)

(21) Appl. No.: 17/760,429

(22) PCT Filed: Feb. 12, 2021
(86) PCT No.: PCT/US21/17990

§ 371 Date: Aug. 9, 2022

Related U.S. Application Data
(60) Provisional application No. 62/975,908, filed on Feb. 13, 2020.

(51) Int. Cl. C07K 16/24; A61K 9/00; A61P 17/00

(52) U.S. Cl. CPC C07K 16/244; A61K 9/0019; A61P 17/00

(57) ABSTRACT

The present disclosure, relates, in general, to methods of treating atopic dermatitis, including moderate or severe atopic dermatitis, and chronic atopic dermatitis, using an antibody specific for thymic stromal lymphopoietin (TSLP).

### CROSS-REFERENCE TO RELATED APPLICATIONS

The present application is a national stage of PCT/US2021/17990, filed Feb. 12, 2021 which claims the priority benefit of U.S. Provisional Patent Application No. 62/975,908, filed Feb. 13, 2020, herein are incorporated by reference in its entirety.

### FIELD OF THE INVENTION

The present disclosure relates, in general, to methods of treating atopic dermatitis, including moderate and severe atopic dermatitis, using an antibody specific for thymic stromal lymphopoietin (TSLP).

Incorporation by Reference of Material Submitted Electronically

Incorporated by reference in its entirety is a computer-readable nucleotide/amino acid sequence listing submitted concurrently herewith and identified as follows: 68,392 byte ASCII (Text) file named “55031_Seqlisting.txt”; created on Aug. 8, 2022.

### BACKGROUND

Atopic dermatitis is a chronic inflammatory skin disease (commonly referred to as eczema). In its severe form, AD is characterized by widespread skin lesions that manifest as red, itchy, swollen, cracked, weeping lesions with crusting and/or scaling. There is frequently intractable pruritus, as well as enhanced susceptibility to bacterial, viral, and fungal skin infections. Atopic dermatitis may affect up to 20% of children and up to 10% of adults (Hanifin and Reed, 2007; Silverberg and Hanifin, 2013; Silverberg et al, 2012). Atopic dermatitis is associated with a substantial patient burden that typically includes poor quality of life, sleep disturbance, and reductions in work productivity (Kiebert et al, 2002). Treatment recommendations for AD include liberal use of emollients, dry skin care protocols, and topical corticosteroids (TCS), which are commonly used in the clinical setting. Unfortunately, TCS have inadequate efficacy in many patients with moderate-to-severe disease, and their long-term use is associated with important side effects, such as thinning and discoloration of the skin, increased risk of skin infection, contact dermatitis, decreased bone density, and slowing of growth in children depending on the extent of percutaneous administration (Chi et al, 2011; Ring et al, 2012).

Thymic stromal lymphopoietin (TSLP), an epithelial cell-derived cytokine produced in response to environmental and pro-inflammatory stimuli, leads to the activation of multiple inflammatory cells and downstream pathways (Soumelis et al., 2002; Allakhverdi et al., J Exp Med 2007; 204:253-8). TSLP is increased in the airways of patients with asthma and correlates with Th2 cytokine and chemokine expression (Shikotra et al. J Allergy Clin Immunol 2012; 129:104-11 e1-9) and disease severity (Ying et al., J Immunol 2005; 174:8183-90; Ying, et al. J Immunol 2008; 181:2790-8). While TSLP is central to the regulation of Th2 immunity, it may also play a key role in other pathways of inflammation and therefore be relevant to multiple disease phenotypes.

Tezepelumab (also known as AMG 157) (Gilliet, et al., J Exp Med 2003; 197:1059-63) is a fully human monoclonal antibody (immunoglobulin G2A) that targets the thymic, stromal lymphopoietin (TSLP), an epithelial-cell-derived cytokine that promotes inflammatory responses to environmental stimuli through its activities on multiple pathways, including (but not limited to) activities on dendritic cells (Gilliet, et al., 2003; Soumelis et al., 2002; Reche, et al. J Immunol 2001; 167:336-43) and mast cells (Allakhverdi et al., 2007). Increased levels of TSLP protein are found in the skin lesions of patients with atopic dermatitis (AD) (Soumelis et al., 2002; Fujisawa et al., J Allergy Clin Immunol 2002; 110:139-46; Hijnen et al., 2004). By binding to TSLP, tezepelumab prevents its interaction with the TSLP receptor complex and inhibits multiple downstream inflammatory pathways.

### SUMMARY

The anti-TSLP antibody described herein addresses an unmet need in atopic dermatitis patients in which other medications may not control moderate to severe atopic dermatitis, including chronic dermatitis. For example, the antibody therapy may improve eczema area and skin lesions in patients, as well as reduce the need for alternate therapies such as topical or systemic corticosteroids.

The disclosure provides a method for treating atopic dermatitis in a subject comprising administering a therapeutically effective amount of an anti-TSLP antibody or antibody variant in a dose of 280 mg to 420 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

Also contemplated is a method for treating atopic dermatitis in a subject comprising administering a therapeutically effective amount of an anti-TSLP antibody or antibody variant in a dose of 280 mg to 420 mg at an interval of every two weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

In various embodiments, the antibody or antibody variant is administered every 4 weeks. In various embodiments, the antibody or antibody variant is administered at a dose of 280 mg or at a dose of 420 mg every 2 weeks or every 4 weeks.

In various embodiments, the antibody or antibody variant is administered at a dose of 280 mg every 2 weeks.

In various embodiments, the antibody or antibody variant is administered at a dose of 420 mg every 2 weeks.

In various embodiments, the subject is also receiving treatment with topical corticosteroids.

In various embodiments, provided is a method for treating atopic dermatitis in a subject comprising administering a therapeutically effective amount of a topical corticosteroid and an anti-TSLP antibody or antibody variant in a dose of 420 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

In various embodiments, provided is a method for treating atopic dermatitis in a subject comprising administering a therapeutically effective amount of a topical corticosteroid and an anti-TSLP antibody or antibody variant in a dose of 280 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

The disclosure also provides a method for treating atopic dermatitis in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

The disclosure further provides a method for treating atopic dermatitis in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

In various embodiments, the anti-TSLP antibody variant has substantially similar pK characteristics as tezepelumab in humans.

In various embodiments, the antibody or antibody variant is administered for a period of at least 4 months, 6 months, 9 months, 1 year or more.

In various embodiments, the anti-TSLP antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody.

In various embodiments, the anti-TSLP antibody or antibody variant thereof is bivalent and selected from the group consisting of a human antibody, a humanized antibody, a chimeric antibody, a monoclonal antibody, a recombinant antibody, an IgG1 antibody, an IgG2 antibody, an IgG3 antibody, and an IgG4 antibody.

In one embodiment, the anti-TSLP antibody variant is selected from the group consisting of a diabody, a triabody, a tetrabody, a Fab fragment, a single domain antibody, an scFv, wherein the dose is adjusted such that the binding sites are equimolar to those dosed by bivalent antibodies.

In various embodiments, the antibody is an IgG2 antibody.

In one embodiment, the antibody or antibody variant is a human antibody.

In various embodiments, the antibody is tezepelumab. In various embodiments, the tezepelumab is an IgG2 antibody having the full length heavy and light chain amino acid sequences set out in SEQ ID NOs: 105 and 106, respectively.

In various embodiments, the antibody or antibody variant further comprises a pharmaceutically acceptable carrier or excipient.

In various embodiments, the atopic dermatitis is moderate atopic dermatitis or severe atopic dermatitis. It is further contemplated that the atopic dermatitis is chronic atopic dermatitis or acute atopic dermatitis. In various embodiments, the atopic dermatitis is lesional atopic dermatitis or nonlesional atopic dermatitis. In various embodiments, the atopic dermatitis is intrinsic or extrinsic dermatitis.

In various embodiments, the antibody is tezepelumab or another anti-TSLP antibody described in the art. Exemplary antibodies are described further in the Detailed Description.

In various embodiments, the subject is an adult. In various embodiments, the subject is a child or adolescent.

It is further contemplated that administration of the anti-TSLP antibody or antibody variant decreases levels of Th2 cytokines in the subject.

In various embodiments, administration of the anti-TSLP antibody or antibody variant improves one or more measures of atopic dermatitis including Investigator's Global Assessment (IGA) score, Eczema Area and Severity Index (EASI) score, Pruritus Numeric Rating Scale (NRS), Patient Oriented Eczema Measure (POEM), Dermatology Quality of Life Index (DLQI), or EuroQOL quality of life 5-dimensions 3-level version (EQ-5D-3L).

In one embodiment, the administration improves one or more symptoms of atopic dermatitis including itching (pruritus), bleeding, oozing, cracked, flaking, and dry/rough skin, impact on sleep, erythema, induration/papulation, excoriation, and lichenification of skin, optionally as measured by atopic dermatitis patient electronic diary.

In various embodiments, the IGA score is reduced by one, two or three points. In various embodiments, the subject has an IGA score of 0 (clear) or 1 (almost clear) (IGA 0/1) at week 16 after treatment. In various embodiments, the subject has a starting IGA score of 2 In various embodiments, the subject has a starting IGA score of 3. In various embodiments, the subject has a starting IGA score of 4. In various embodiments, the subject has a starting IGA score of 5.

In various embodiments, the EASI is reduced by 10%, 15%, 25%, 30%, 35%, 40%, 45%, 50%, 55%, 60%, 65%, 70%, 75%, 80%, 85%, 90% or more. In various embodiments there is a 75% reduction in EASI (EASI 75) at week 16 after treatment. In various embodiments, EASI is reduced 20% to 70% (EASI 50/90) at week 12 after treatment compared to baseline or placebo group, or EASI is reduced 50% to 90% (EASI 50/90) at week 16 after treatment compared to baseline or placebo group. In various embodiments, the time needed to reach EASI 50/75/90 is reduced in subjects receiving anti-TSLP antibody compared to subjects not receiving anti-TSLP.

In various embodiments, the SCORAD score of the subject is reduced by 20%, 25%, 30%, 35%, 40%, 45%, 50%, 55%, 60%, 65%, 70%, 75%, 80%, 85%, 90% or more compared to subjects not receiving anti-TSLP.

In various embodiments, treatment with anti-TSLP modulates the levels of one or more biomarkers of atopic dermatitis, including, cytokines, IgE, CCL17, CCL18, CCL22, and RNA transcriptional changes in blood and lesional versus nonlesional skin. In various embodiments, treatment with anti-TSLP reduces the level of Th2 cytokines. In various embodiments, the treatment modulates (reduces or moderates) levels of or activity of one or more of IL-4, IL-5, IL-13, IL-17, IL-22, IL-23, IL-31, IL-33, or combinations thereof.

Further provided is a method for treating atopic dermatitis in a subject comprising administering a therapeutically effective amount of an anti-TSLP antibody or antibody variant in a dose of 280 to 420 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2, wherein the antibody is an IgG2 antibody.

In various embodiments, the antibody or antibody variant is administered every 4 weeks. In various embodiments, the antibody or antibody variant is administered at a dose of 280 mg or at a dose of 420 mg every 2 weeks or every 4 weeks.

In various embodiments, the antibody or antibody variant is administered at a dose of 280 mg every 2 weeks.

In various embodiments, the antibody or antibody variant is administered at a dose of 420 mg every 2 weeks.

In various embodiments, the antibody or antibody variant is administered at a dose of 210 mg every 4 weeks.

Also provided is a method of reducing the frequency of atopic dermatitis exacerbation in a subject comprising administering a therapeutically effective amount of an anti-TSLP antibody or antibody variant in a dose of 280 mg to 420 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

Further contemplated is a method of reducing the frequency of atopic dermatitis exacerbation in a subject comprising administering a therapeutically effective amount of an anti-TSLP antibody or antibody variant in a dose of 280 mg to 420 mg at an interval of every 2 weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

Also provided is a method of reducing the frequency of atopic dermatitis exacerbation in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

Also provided is a method of reducing the frequency of atopic dermatitis exacerbation in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

Also provided are methods for reducing Investigator's Global Assessment (IGA) and/or Eczema Area and Severity Index (EASI) score in a subject comprising administering a therapeutically effective amount of an anti-TSLP antibody or antibody variant in a dose of 280 mg to 420 mg at an interval of every 2 weeks, or at a dose of 210 mg at an interval or every 4 weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

In various embodiments, the disclosure provides a method for reducing Investigator's Global Assessment (IGA) and/or Eczema Area and Severity Index (EASI) score in a subject comprising administering a therapeutically effective amount of an anti-TSLP antibody or antibody variant in a dose of 280 mg to 420 mg at an interval of every 2 weeks, or at a dose of 210 mg at an interval or every 4 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

In various embodiments, both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

Also contemplated is a method for treating obstructive pulmonary disease (COPD) in a subject comprising administering a therapeutically effective amount of an anti-TSLP antibody or antibody variant in a dose of 280 mg to 420 mg at an interval of every 2 weeks, wherein both binding sites of the antibody have identical binding to TSLP, and the antibody comprises a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

Also provided is a method for treating obstructive pulmonary disease (COPD) in a subject comprising administering a therapeutically effective amount of an anti-TSLP antibody or antibody variant in a dose of 280 mg to 420 mg at an interval of every two weeks, wherein both binding sites of the antibody have identical binding to TSLP, and the antibody comprises a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

It is contemplated that the dosing and antibody and antibody variant types referenced above apply to each method contemplated herein.

In various embodiments, the antibody or antibody variant further comprises a pharmaceutically acceptable carrier or excipient.

In various embodiments, the administration delays the time to an atopic dermatitis exacerbation compared to a subject not receiving the anti-TSLP antibody.

In various embodiments, the administration reduces frequency of or levels of co-administered therapy in the subject. Optionally, the co-administered therapy is topical corticosteroids, topical calcineurin inhibitors, dupilumab, immunosuppressive or immunomodulating drugs (e.g., systemic corticosteroids, cyclosporine, mycophenolate-mofetil, interferon (IFN)-gamma, Janus kinase inhibitors, azathioprine, methotrexate), anti-IL-13 antibodies, anti-IL-5 pathway antibodies (benralizumab, mepolizumab, reslizumab), or combinations thereof.

In various embodiments, the administration eliminates the need for corticosteroid therapy.

In various embodiments, the administration is subcutaneous or intravenous.

In various embodiments, the antibody is tezepelumab or another anti-TSLP antibody described in the art, e.g., in Table A. Exemplary antibodies are described further in the Detailed Description.

### BRIEF DESCRIPTION OF THE DRAWINGS

FIG. 1 depicts the overall EASI 75 response rates for all dose groups at week 16.

FIG. 2 shows a comparison of EASI scores over 16 weeks or 12 weeks of treatment for the different treatment groups.

FIG. 3 illustrates the response rate observed in moderate AD (BL EASI 21) relative to severe AD.

### DETAILED DESCRIPTION

Use of an anti-TSLP antibody addresses an unmet need in atopic dermatitis patients in which other medications may not control moderate to severe atopic dermatitis. It is further contemplated that treatment with anti-TSLP antibodies such as tezepelumab could eliminate regular disease activity and make more patients steroid-free or reduce the need for use of steroids in the treatment of atopic dermatitis.

### Definitions

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

Unless otherwise defined, all technical and scientific terms used herein have the same meaning as commonly understood by one of ordinary skill in the art to which the present disclosure belongs. The following references provide one of skill with a general definition of many of the terms used in this disclosure include, but are not limited to: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “atopic dermatitis” as used herein refers to an inflammatory skin disease that can be chronic or acute, and can range in severity from moderate to severe. Atopic dermatitis includes intrinsic or extrinsic dermatitis, depending on the level of IgE antibody present in patient samples.

The term “atopic dermatitis exacerbation” as used herein refers to a worsening of atopic dermatitis that leads to any of the following: Use of topical or systemic corticosteroids or other adjunct therapy for at least 3 days; worsening of one or more symptoms or measures of atopic dermatitis described herein, and/or growth or expansion of lesion size or severity.

The term “worsening of atopic dermatitis” refers to new or increased symptoms and/or signs (examination or skin lesion or atrophy) that can be either concerning to the subject (subject-driven) or related to an Patient Daily Diary alert (diary-driven).

The term “cytokine” as used herein refers to one or more small (5-20 kD) proteins released by cells that have a specific effect on interactions and communications between cells or on the behavior of cells, such as immune cell proliferation and differentiation. Functions of cytokines in the immune system include, promoting influx of circulating leukocytes and lymphocytes into the site of immunological encounter; stimulating the development and proliferation of B cells, T cells, peripheral blood mononuclear cells (PBMCs) and other immune cells; and providing antimicrobial activity. Exemplary immune cytokines, include but are not limited to, IL-1, IL-2, IL-3, IL-4, IL-5, IL-6, IL-7, IL-9, IL-10, IL-12, IL-13, IL-15, IL17A, IL-17F, IL-18, IL-21, IL-22, IL-23, IL-31, IL-33, interferon (including IFN alpha, beta, and gamma), tumor necrosis factor (including TNF alpha, beta), transforming growth factor (including TGF alpha, beta), granulocyte colony stimulating factor (GCSF), granulocyte macrophage colony stimulating factor (GMCSF) and thymic stromal lymphopoietin (TSLP).

A “T helper (Th) 1 cytokine” or “Th1-specific cytokine” refers to cytokines that are expressed (intracellularly and/or secreted) by Th1 T cells, and include IFN-g, TNF-α, and IL-12. A “Th2 cytokine” or “Th2-specific cytokine” refers to cytokines that are expressed (intracellularly and/or secreted) by Th2 T cells, including IL-4, IL-5, IL-13, and IL-10. A “Th17 cytokine” or “Th17-specific cytokine” refers to cytokines that are expressed (intracellularly and/or secreted) by Th17 T cells, including IL-17A, IL-17F, IL-22 and IL-21. Certain populations of Th17 cells express IFN-g and/or IL-2 in addition to the Th17 cytokines listed herein. A polyfunctional CTL cytokine includes IFN-g, TNF-α, IL-2 and IL-17.

The term “specifically binds” is “antigen specific”, is “specific for”, “selective binding agent”, “specific binding agent”, “antigen target” or is “immunoreactive” with an antigen refers to an antibody or polypeptide that binds an target antigen with greater affinity than other antigens of similar sequence. It is contemplated herein that the agent specifically binds target proteins useful in identifying immune cell types, for example, a surface antigen (e.g., T cell receptor, CD3), a cytokine (e.g., TSLP, IL-4, IL-5, IL-13, IL-17, IFN-g, TNF-α) and the like. In various embodiments, the antibody specifically binds the target antigen, but can cross-react with an ortholog of a closely related species, e.g. an antibody may being human protein and also bind a closely related primate protein.

The term “antibody” or “immunoglobulin” refers to a tetrameric glycoprotein that consists of two heavy chains and two light chains, each comprising a variable region and a constant region. “Heavy Chains” and “Light Chains” refer to substantially full length canonical immunoglobulin light and heavy chains (see e.g., Immunobiology, 5th Edition (Janeway and Travers et al., Eds., 2001). Antigen-binding portions may be produced by recombinant DNA techniques or by enzymatic or chemical cleavage of intact antibodies. The term “antibody” includes monoclonal antibodies, polyclonal antibodies, chimeric antibodies, human antibodies, and humanized antibodies.

Antibody variants include antibody fragments and anti-body like proteins with changes to structure of canonical tetrameric antibodies. Typically antibody variants include V regions with a change to the constant regions, or, alternatively, adding V regions to constant regions, optionally in a non-canonical way. Examples include multispecific antibodies (e.g., bispecific antibodies with extra V regions), antibody fragments that can bind an antigen (e.g., Fab′, F′(ab)2, Fv, single chain antibodies, diabodies), biparatopic and recombinant peptides comprising the forgoing as long as they exhibit the desired biological activity.

Antibody fragments include antigen-binding portions of the antibody including, inter alia, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Valency” refers to the number of antigen binding sites on each antibody or antibody fragment that targets an epitope. A typical full length IgG molecule, or F(ab)₂ is “bivalent” in that it has two identical target binding sites. A “monovalent” antibody fragment such as a F(ab)′ or scFc with a single antigen binding site. Trivalent or tetravalent antigen binding proteins can also be engineered to be multivalent.

“Monoclonal antibody” refers to an antibody obtained from a population of substantially homogeneous antibodies, i.e., the individual antibodies comprising the population are identical except for possible naturally occurring mutations that may be present in minor amounts.

The term “inhibits TSLP activity” includes inhibiting any one or more of the following: binding of TSLP to its receptor; proliferation, activation, or differentiation of cells expressing TSLPR in the presence of TSLP; inhibition of Th2 cytokine production in a polarization assay in the presence of TSLP; dendritic cell activation or maturation in the presence of TSLP; and mast cell cytokine release in the presence of TSLP. See, e.g., U.S. Pat. No. 7,982,016 B2, column 6 and example 8 and US 2012/0020988 A1, examples 7-10.

The term “sample” or “biological sample” refers to a specimen obtained from a subject for use in the present methods, and includes urine, whole blood, plasma, serum, saliva, sputum, skin or tissue biopsies, cerebrospinal fluid, peripheral blood mononuclear cells with in vitro stimulation, peripheral blood mononuclear cells without in vitro stimulation, gut lymphoid tissues with in vitro stimulation, gut lymphoid tissues without in vitro stimulation, gut lavage, bronchioalveolar lavage, nasal lavage, and induced sputum.

The terms “treat”, “treating” and “treatment” refer to eliminating, reducing, suppressing or ameliorating, either temporarily or permanently, either partially or completely, a clinical symptom, manifestation or progression of an event, disease or condition associated with an inflammatory disorder described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic agent in an amount and for a time sufficient to induce a sustained improvement over baseline of an indicator that reflects the severity of the particular disorder.

The term “therapeutically effective amount” refers to an amount of therapeutic agent that is effective to ameliorate or lessen symptoms or signs of disease associated with a disease or disorder.

Atopic Dermatitis

Atopic dermatitis (AD) is a chronic inflammatory skin disease (commonly referred to as eczema). In its severe form, AD is characterized by widespread skin lesions that manifest as red, itchy, swollen, cracked, weeping lesions with crusting and/or scaling.

The immunopathology of AD varies depending on the type of skin lesions. Acute lesions are characterized by a robust T helper 2 (Th2) immune response with production of interleukin (IL)-4, IL-5, and IL-13; and release of the epithelial cytokines IL-33, IL-25, and TSLP. Chronic lesions are characterized by a mixed Th1 and Th2 response (Beck and Leung, 2000; Soumelis et al, 2002).

Although TSLP is not detectable in the nonlesional skin of patients with AD, TSLP levels are elevated in both acute and chronic AD lesions (Soumelis et al, 2002). Thymic stromal lymphopoietin increases the number and maturation status of human migratory Langerhans cells and, through dendritic cells, induces a Th2 cytokine profile in cluster of differentiation (CD) 4+ helper T cells (Ebner et al, 2007). In a mouse model of AD, TSLP has been shown to directly stimulate antigen-specific CD4+T cells to produce a Th2 cytokine profile following cutaneous allergen challenge (He et al, 2008). The role of TSLP in the development of fibrosis in AD is not well understood, but TSLP is detectable in lung fibroblasts (Soumelis et al, 2002), and a recent study suggests that TSLP directly promotes fibrocytes to produce collagen and that neutralization of TSLP significantly reduces skin fibrosis (Oh et al, 2011). As further evidence for the role of TSLP in the initiation and progression of AD, studies in mice overexpressing epidermal-specific TSLP demonstrate clinical and cellular features similar to human AD, including the development of eczematous lesions containing inflammatory cell infiltrates. Furthermore, in these studies, mice lacking T cells still develop AD-like disease, suggesting that TSLP can trigger AD-like skin inflammation in mice through direct activation of myeloid cells (Yoo et al, 2005).

Although most patients develop AD before the age of 5 years, up to 20% of patients may not develop AD until adulthood (Bieber et al, 2002; Kim et al, 2016). For many children, the onset of AD marks the beginning of a progression from AD to food allergy, allergic rhinitis, and eventually asthma, a phenomenon frequently described as the “atopic march” (Spergel and Paller, 2003). Indeed, up to 70% of patients with severe AD will develop asthma (Zheng et al, 2011).

Numerous studies support a role for TSLP across the spectrum of atopic diseases and suggest that TSLP may function not only as an upstream regulator of inflammation in AD but as a modulator of disease progression within the atopic march. Zhang et al. used a mouse model to demonstrate that induced expression of TSLP in mouse epidermal keratinocytes not only triggers AD but also induces experimental allergic asthma (Zhang et al, 2009). In studies by Demehri and colleagues, mice overexpressing TSLP from skin keratinocytes not only developed AD-like skin inflammation but also were also susceptible to allergen-induced asthma. Furthermore, these authors demonstrated that epidermal-derived TSLP was sufficient to confer a severe asthma phenotype even in the absence of any skin defect (Demehri et al, 2009). Taken together, these studies suggest that TSLP is both necessary and sufficient to trigger the atopic march in a mouse model. In support of a key role for TSLP in the early development of AD, a prospective human birth cohort study revealed that high TSLP levels in skin at 2 months of age in addition to family history is a better predictor of AD development later in life compared to family history alone (Kim et al, 2016).

AD can be assessed using several objective and subjective measurements such as the Investigator's Global Assessment (IGA) and Eczema Area and Severity Index (EASI). The IGA allows investigators to assess overall disease severity at one given time point and consists of a 5-point severity scale from clear to severe disease (0=clear; 1=almost clear; 2=mild disease; 3=moderate disease; 4=severe disease; 5=very severe disease). The IGA uses clinical characteristics of erythema, infiltration, papulation, oozing, and crusting as guidelines for the overall severity assessment (Breuer et al, 2004).

The EASI was designed by modifying the Psoriasis Area and Severity Index, which has been widely used in clinical trials and has been established as a well-accepted and standardized instrument for assessing therapeutic response in patients with psoriasis (Schmitt et al, 2007). The EASI evaluates 4 natural anatomical regions for severity and extent of key disease signs and focuses on key acute and chronic signs of inflammation (i.e., erythema, induration/papulation, excoriation, and lichenification). The maximum score is 72, with higher values indicating more severe disease.

Additional measures of AD severity/improvement include Scoring Atopic Dermatitis (SCORAD), the patient reported outcome (PRO) measure of pruritus assessed using a numeric rating scale (NRS) (Pruritus NRS); and biomarkers such as IgE, CCL17, CCL22, and RNA transcriptional changes in blood and lesional versus nonlesional skin, Patient Global Impression of Severity (PGI-S), and health-related quality of life (HRQoL) assessed using the Dermatology Life Quality Index (DLQI).

SCORAD is a clinical tool for assessing the severity (i.e., extent, intensity) of AD. SCORAD evaluates the extent and intensity of the AD lesions, along with subjective symptoms (Kunz et al, 1997). The maximum total score is 103, with higher values indicating more severe disease. Patient-reported Outcomes (PRO) includes the electronic diary (eDiary), which includes the pruritus NRS. Pruritus is assessed using an numeric rating scale (NRS) (0-10) with 0=no itch and 10=worst imaginable itch.

The Patient Oriented Eczema Measure (POEM) is a 7-item validated questionnaire used in clinical practice and clinical trials to assess disease symptoms in children and adults (Charman, 2004). It evaluates time spent in the past week with AD signs and symptoms: individual items of itching, bleeding, oozing, cracked, flaking, and dry/rough skin, and their impact on sleep.

The Dermatology Quality of Life Index (DLQI) is a 10-item, subject-completed, HRQoL assessment with content specific to those with dermatology conditions. The recall period is 1 week (Finlay and Kahn, 1994). The DLQI content captures respondent perceptions of dermatology-related symptoms and feelings (embracement), impacts on daily activities, leisure, work or school, personal relationships, and the side effects of treatment. Each item is scored on a 4-point Likert scale: 0=not at all/not relevant; 1=a little; 2=a lot; and 3=very much (Basra et al, 2008).

The EuroQOL quality of life 5-dimensions 3-level version (EQ-5D-3L) is a standardized instrument for use as a measure of health-related quality of life (HRQoL) and was developed by EuroQol (Brooks, 1996). It defines health in terms of 5 dimensions: mobility, self-care, usual activities, pain/discomfort, and anxiety/depression. Each dimension has 3 ordinal levels of severity: 1, no problem; 2, some problems; and 3, severe problems. Overall health state is defined as a 5-digit number.

The Patient Global Impression of Severity (PGI-S) is a single item designed to capture the subject's perception of overall symptom severity at the time of completion on a 5-point categorical response scale (no symptoms to very severe symptoms).

It is contemplated that treatment with anti-TSLP improves one or more of the AD symptoms or measurements described herein, such as Investigator's Global Assessment (IGA) score, Eczema Area and Severity Index (EASI) score, Pruritus Numeric Rating Scale, Patient Oriented Eczema Measure, Dermatology Quality of Life Index (DLQI), EuroQOL quality of life 5-dimensions 3-level version (EQ-5D-3L), itching (pruritus), bleeding, oozing, cracked, flaking, and dry/rough skin, impact on sleep, erythema, induration/papulation, excoriation, and lichenification of skin, optionally as measured by atopic dermatitis patient electronic diary.

### TSLP

Thymic stromal lymphopoietin (TSLP) is an epithelial cell-derived cytokine that is produced in response to pro-inflammatory stimuli and drives allergic inflammatory responses primarily through its activity on dendritic cells (Gilliet, J Exp Med. 197:1059-1067, 2003; Soumelis, Nat Immunol. 3:673-680, 2002; Reche, J Immunol. 167:336-343, 2001), mast cells (Allakhverdi, J Exp Med. 204:253-258, 2007) and CD34+progenitor cells.⁹ TSLP signals through a heterodimeric receptor consisting of the interleukin (IL)-7 receptor alpha (IL-7Ra) chain and a common y chain-like receptor (TSLPR) (Pandey, Nat Immunol. 1:59-64, 2000; Park, J Exp Med. 192:659-669, 2000).

Data from other studies suggest that TSLP may promote airway inflammation through Th2 independent pathways such as the crosstalk between airway smooth muscle and mast cells (Allakhverdi et al, J Allergy Clin Immunol. 123(4):958-60, 2009; Shikotra et al, supra). TSLP can also promote induction of T cells to differentiate into Th-17-cytokine producing cells with a resultant increase in neutrophilic inflammation commonly seen in more severe asthma (Tanaka et al, Clin Exp Allergy. 39(1):89-100, 2009). These data and other emerging evidence suggest that blocking TSLP may serve to suppress multiple biologic pathways including but not limited to those involving Th2 cytokines (IL-4/13/5).

### Antibodies

It is contemplated that antibodies or antibody variants specific for TSLP are useful in the treatment of atopic dermatitis, including moderate or severe atopic dermatitis, chronic or acute atopic dermatitis, lesional or nonleasional atopic dermatitis, and other forms of atopic dermatitis.

Specific binding agents such as antibodies and antibody variants or fragments that bind to their target antigen, e.g., TSLP, are useful in the methods of the disclosure. In one embodiment, the specific binding agent is an antibody. The antibodies may be monoclonal (MAbs); recombinant; chimeric; humanized, such as complementarity-determining region (CDR)-grafted; human; antibody variants, including single chain; and/or bispecific; as well as fragments; variants; or derivatives thereof. Antibody fragments include those portions of the antibody that bind to an epitope on the polypeptide of interest. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Monoclonal antibodies may be modified for use as therapeutics or diagnostics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody.

Also encompassed by the disclosure are human antibodies and antibody variants (including antibody fragments) that bind TSLP. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 546073B1 and 546073A1. Human antibodies can also be produced by the expression of recombinant DNA in host cells or by expression in hybridoma cells as described herein.

Chimeric, CDR grafted, and humanized antibodies and/or antibody variants are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

Antibodies and antibody variants (including antibody fragments) useful in the present methods comprise an anti-TSLP antibody comprising a. a light chain variable domain comprising: i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and

b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8, wherein the antibody or antibody variant specifically binds to a TSLP polypeptide as set forth in amino acids 29-159 of SEQ ID NO:2.

Also contemplated is an antibody or antibody variant comprising a. a light chain variable domain selected from the group consisting of: i. a sequence of amino acids at least 80% identical to SEQ ID NO:12; ii. a sequence of amino acids encoded by a polynucleotide sequence that is at least 80% identical to SEQ ID NO:11; iii. a sequence of amino acids encoded by a polynucleotide that hybridizes under moderately stringent conditions to the complement of a polynucleotide consisting of SEQ ID NO:11; and

b. a heavy chain variable domain selected from the group consisting of: i. a sequence of amino acids that is at least 80% identical to SEQ ID NO:10; ii. a sequence of amino acids encoded by a polynucleotide sequence that is at least 80% identical to SEQ ID NO:9; iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody or antibody variant specifically binds to a TSLP polypeptide as set forth in amino acids 29-159 of SEQ ID NO:2.

Tezepelumab is an exemplary anti-TSLP antibody having: a. i. a light chain CDR1 sequence comprising the amino acid sequence set forth in SEQ ID NO:3; ii. a light chain CDR2 sequence comprising the amino acid sequence set forth in SEQ ID NO:4; iii. a light chain CDR3 sequence comprising the amino acid sequence set forth in SEQ ID NO:5; and b. a heavy chain variable domain comprising: i. a heavy chain CDR1 sequence comprising the amino acid sequence set forth in SEQ ID NO:6; ii. a heavy chain CDR2 sequence comprising the amino acid sequence set forth in SEQ ID NO:7, and iii. a heavy chain CDR3 sequence comprising the amino acid sequence set forth in SEQ ID NO:8;

Tezepelumab also comprises a light chain variable domain having the amino acid sequence set out in SEQ ID NO:12; encoded by a polynucleotide sequence set out in SEQ ID NO:11; and a heavy chain variable domain having the amino acid sequence set out in SEQ ID NO:10, encoded by a polynucleotide sequence set out in SEQ ID NO:9.

Tezepelumab is an IgG2 antibody. The sequence of the full length heavy chain and light chain of tezepelumab, including the IgG2 chain, is set out in SEQ ID NOs: 105 and 106, respectively.

In various embodiments, the anti-TSLP antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody.

In various embodiments, the anti-TSLP antibody or antibody variant thereof is bivalent and selected from the group consisting of a human antibody, a humanized antibody, a chimeric antibody, a monoclonal antibody, a recombinant antibody, an IgG1 antibody, an IgG2 antibody, an IgG3 antibody, and an IgG4 antibody.

In various embodiments, the anti-TSLP antibody variant is selected from the group consisting of a diabody, a triabody, a tetrabody, a Fab fragment, single domain antibody, scFv, wherein the dose is adjusted such that the binding sites to be equimolar to the those dosed by bivalent antibodies.

It is contemplated that the antibody or antibody variant is an IgG2 antibody. Exemplary sequences for a human IgG2 constant region are available from the Uniprot database as Uniprot number P01859, incorporated herein by reference. Information, including sequence information for other antibody heavy and light chain constant regions is also publicly available through the Uniprot database as well as other databases well-known to those in the field of antibody engineering and production.

In certain embodiments, derivatives of antibodies include tetrameric glycosylated antibodies wherein the number and/or type of glycosylation site has been altered compared to the amino acid sequences of a parent polypeptide. In certain embodiments, variants comprise a greater or a lesser number of N-linked glycosylation sites than the native protein. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to human TSLP, or to increase or decrease the affinity of the antibodies to human TSLP described herein.

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

### Methods of Administration

In one aspect, methods of the present disclosure include a step of administering a therapeutic anti-TSLP antibody or antibody variant described herein, optionally in a pharmaceutically acceptable carrier or excipient. In certain embodiments, the pharmaceutical composition is a sterile composition.

Contemplated herein are methods method for treating atopic dermatitis in a subject, including severe or moderate atopic dermatitis, chronic or acute atopic dermatitis, or lesional or nonlesional atopic dermatitis.

In various embodiments, the antibody is tezepelumab or another anti-TSLP antibody described in the art. Exemplary anti-TSLP antibodies include antibodies described in WO 2017/042701, WO 2016/142426, WO 2010/017468, US20170066823, US20120020988 and U.S. Pat. No. 8,637,019, incorporated herein by reference, some of which are described below in Table A. In exemplary aspects, the anti-TSLP antibody is selected from an antibody of Table A.

TABLE A

| | |
|---|---|
| W02017/042701 | An anti-TSLP antibody comprising a heavy chain (HC) CDR1 comprising |
| | the sequence of SEQ ID NO: 13, a HC CDR2 comprising the sequence |
| | of SEQ ID NO: 14, and a HC CDR3 comprising the sequence of SEQ ID |
| | NO: 15; |
| | An anti-TSLP antibody comprising a light chain (LC) CDR1 comprising |
| | the sequence of SEQ ID NO: 16, a LC CDR2 comprising the sequence |
| | of SEQ ID NO: 17, a LC CDR3 comprising the sequence of SEQ ID NO: |
| | 18; |
| | An anti-TSLP antibody comprising a heavy chain (HC) CDR1 comprising |
| | the sequence of SEQ ID NO: 19, a HC CDR2 comprising the sequence |
| | of SEQ ID NO: 20, a HC CDR3 comprising the sequence of SEQ ID |
| | NO: 15; |
| | An anti-TSLP antibody comprising a light chain (LC) CDR1 comprising |
| | the sequence of SEQ ID NO: 21, a LC CDR2 comprising the sequence |
| | of SEQ ID NO: 22, a LC CDR3 comprising the sequence of SEQ ID NO: |
| | 23; |
| | An anti-TSLP antibody comprising a HC variable region comprising the |
| | sequence of SEQ ID NO: 26 and/or a LC variable region comprising the |
| | sequence of SEQ ID NO: 27; |
| | An anti-TSLP antibody comprising a HC variable region comprising the |
| | sequence of SEQ ID NO: 28 and/or a LC variable region comprising the |
| | sequence of SEQ ID NO: 29; |
| | An anti-TSLP antibody that comprises a paratope comprising at least |
| | one of the following residues: Thr28, Asp31, Tyr32, Trp33, Asp56, |
| | Glu101, Ile102, Tyr103, Tyr104, Tyr105 of a heavy chain sequence of |
| | SEQ ID NO: 26 or Gly28, Ser29, Lys30, Tyr31, Tyr48, Asp50, Asn51, |
| | Glu52, Asn65, and Trp92 of a light chain sequence of SEQ ID NO: 27; |
| | An anti-TSLP antibody that specifically binds an epitope in human |
| | TSLP, wherein the epitope comprises at least one of the following |
| | residues: Lys38, Ala41, Leu44, Ser45, Thr46, Ser48, Lys49, Ile52, |
| | Thr53, Ser56, Gly57, Thr58, Lys59, Lys101, Gln145, and Arg149 of |
| | SEQ ID NO: 30; |
| WO2016/142426 | An anti-TSLP antibody comprising the amino acid sequence of SEQ ID |
| | NO: 31; |
| | An anti-TSLP antibody comprising a CDR1 comprising the sequence of |
| | SEQ ID NO: 32; a CDR2 comprising the sequence of SEQ ID NO: 33, |
| | and a CDR3 comprising the sequence of SEQ ID NO: 34; |
| | An anti-TSLP antibody comprising a CDR1 comprising the sequence of |
| | SEQ ID NO: 32; a CDR2 comprising the sequence of SEQ ID NO: 35, |
| | and a CDR3 comprising the sequence of SEQ ID NO: 34; |
| | An anti-TSLP antibody comprising a variant of the CDR1 of SEQ ID NO: |
| | 31 wherein the residue corresponding to residue 28 in SEQ ID NO: 31 is |
| | Pro, the residue corresponding to residue 30 in SEQ ID NO: 31 is Arg, |
| | the residue corresponding to residue 31 in SEQ ID NO: 31 is Asn, the |
| | residue corresponding to residue 32 in SEQ ID NO: 31 is Trp and the |
| | residue corresponding to residue 34 in SEQ ID NO: 31 is Asp; |
| | An anti-TSLP antibody comprising a variant of the CDR2 of SEQ ID NO: |
| | 31 wherein the residue corresponding to residue 50 in SEQ ID NO: 31 is |
| | Gly, the residue corresponding to residue 53 in SEQ ID NO: 31 is His |
| | and the residue corresponding to residue 55 in SEQ ID NO: 31 is Gin; |
| | An anti-TSLP antibody comprising a variant of the CDR3 of SEQ ID NO: |
| | 31 wherein the residue corresponding to residue 91 in SEQ ID NO: 31 is |
| | He, Leu, Val or Phe, the residue corresponding to residue 92 in SEQ ID |
| | NO: 31 is Gly or Ala, the residue corresponding to residue 93 in SEQ ID |
| | NO: 31 is Glu, Phe, Asp or Ser and the residue corresponding to residue |
| | 94 in SEQ ID NO: 31 is Asp. |
| WO2010/017468 | An anti-TSLP antibody (9B7) comprising a HC CDR3 comprising the |
| | sequence of SEQ ID NO: 38, wherein the other CDRs of the HC and LC |
| | comprise the sequences of SEQ ID NOs: 36, 37, and 39-41; |
| | An anti-TSLP antibody (6C5) comprising a HC CDR3 comprising the |
| | sequence of SEQ ID NO: 44, wherein the other CDRs of the HC and LC |
| | comprise the sequences of SEQ ID NOs: 42, 43, and 45-47; |
| | An anti-TSLP antibody (6A3) comprising a HC CDR3 comprising the |
| | sequence of SEQ ID NO: 50, wherein the other CDRs of the HC and LC |
| | comprise the sequences of SEQ ID NOs: 48, 49, and 51-53; |
| | An anti-TSLP antibody (1A11) comprising a HC CDR3 comprising the |
| | sequence of SEQ ID NO:56, wherein the other CDRs of the HC and LC |
| | comprise the sequences of SEQ ID NOs: 54, 55, and 57-59; |
| | An anti-TSLP antibody comprising (i) heavy chain variable region of |
| | SEQ ID NO: 60 and/or the light chain variable region of SEQ ID NO: 61; |
| | An anti-TSLP antibody comprising (i) heavy chain variable region of |
| | SEQ ID NO: 62 and/or the light chain variable region of SEQ ID NO: 63; |
| | An anti-TSLP antibody comprising (i) heavy chain variable region of |
| | SEQ ID NO: 64 and/or the light chain variable region of SEQ ID NO: 65; |
| | An anti-TSLP antibody comprising (i) heavy chain variable region of |
| | SEQ ID NO: 66 and/or the light chain variable region of SEQ ID NO: 67; |
| | An anti-TSLP antibody comprising (i) heavy chain variable region of |
| | SEQ ID NO: 68 and/or the light chain variable region of SEQ ID NO: 69; |
| | An anti-TSLP antibody comprising a HC CDR selected from the group |
| | consisting of SEQ ID NO: 38, SEQ ID NO: 44, SEQ ID NO: 50 and SEQ |
| | ID NO: 56, and analogs thereof; |
| | An anti-TSLP antibody comprising a heavy chain comprising the |
| | following CDRs or analogs thereof CDRH1: RYNVH (SEQ ID NO: 36), |
| | CDRH2: MIWDGGSTDYNSALKS (SEQ ID NO: 37), CDRH3: NRYESG |
| | (SEQ ID NO: 38), and a light chain comprising the following CDRs or |
| | analogs thereof CDRL1: KSSQSLLNSGNRKNYLT (SEQ ID NO: 39), |
| | CDRL2: WASTRES (SEQ ID NO: 40), and CDRL3: QNDYTYPFTFGS |
| | (SEQ ID NO: 41); or |
| | An anti-TSLP antibody comprising a heavy chain comprising the |
| | following CDRs or analogs thereof CRDH1: AYWMS (SEQ ID |
| | NO: 42), CDRH2: EINPDSSTINCTPSLKD (SEQ ID NO: 43), CDRH3: |
| | RLRPFWYFDVW (SEQ ID NO: 44), and a light chain comprising the |
| | following CDRs or analogs thereof CDRL1: RSSQSIVQSNGNTYLE |
| | (SEQ ID NO: 45), CDRL2: KVSNRFS (SEQ ID NO: 46), and CDRL3: |
| | FQGSHVPRT (SEQ ID NO: 47); |
| | An anti-TSLP antibody comprising a heavy chain comprising the |
| | following CDRs or analogs thereof CRDH1: TDYAWN (SEQ ID NO: 48), |
| | CDRH2: YIFYSGSTTYTPSLKS (SEQ ID NO: 49), CDRH3: GGYDVNYF |
| | (SEQ ID NQ: 50), and a light chain comprising the following CDRs or |
| | analogs thereof CDRL1: LASQTIGAWLA (SEQ ID NO: 51), CDRL2: |
| | AATRLAD (SEQ ID NO: 52), and CDRL3: QQFFSTPWT (SEQ ID |
| | NO: 53); |
| | An anti-TSLP antibody comprising a heavy chain comprising the |
| | following CDRs or analogs thereof CDRH1 : GYTMN (SEQ ID NO: 54), |
| | CDRH2: LINPYNGVTSYNQKFK (SEQ ID NO: 55), CDRH3: |
| | GDGNYWYF (SEQ ID NO: 56), and a light chain comprising the |
| | following CDRs or analogs thereof CDRL1: SASSSVTYMHW (SEQ ID |
| | NO: 57), CDRL2: EISKLAS (SEQ ID NO: 58), and CDRL3: QEWNYPYTF |
| | (SEQ ID NO: 59); |
| | An anti-TSLP antibody comprising a HC CDR1 comprising the sequence |
| | of SEQ ID NO: 70; a CDR2 comprising the sequence of SEQ ID NO: 71, |
| | and a CDR3 comprising the sequence of SEQ ID NO: 72; |
| | An anti-TSLP antibody comprising a LC CDR1 comprising the sequence |
| | of SEQ ID NO: 73; a CDR2 comprising the sequence of SEQ ID NO: 74, |
| | and a CDR3 comprising the sequence of SEQ ID NO: 75; |
| US2012/0020988 | An anti-TSLP antibody comprising a heavy chain variable domain |
| | comprising a CDR1 region of SEQ ID NO: 76, a CDR2 region of SEQ ID |
| | NO: 77, and CDR3 region of SEQ ID NO: 78, and a light chain variable |
| | domain comprising a CDR1 region of SEQ ID NO: 79, a CDR2 region of |
| | SEQ ID NO: 80, and a CDR3 region of SEQ ID NO: 81. |
| | An anti-TSLP antibody comprising a heavy chain variable domain |
| | comprising SEQ ID NO: 82 and a light chain variable domain comprising |
| | SEQ ID NO: 83; |
| | An anti-TSLP antibody comprising a heavy chain variable domain |
| | comprising a CDR1 region of SEQ ID NO: 76 or 84, a CDR2 region of |
| | SEQ ID NO: 77 or 85, and CDR3 region of SEQ ID NO: 78, and a light |
| | chain variable domain comprising a CDR1 region of SEQ ID NO: 79 or |
| | 86, a CDR2 region of SEQ ID NO: 80, 87, or 88, and a CDR3 region of |
| | SEQ ID NO: 81. |
| | An anti-TSLP antibody comprising a heavy chain variable domain |
| | comprising a CDR1 region of SEQ ID NO: 76, a CDR2 region of SEQ ID |
| | NO:85, and CDR3 region of SEQ ID NO: 78, and a light chain variable |
| | domain comprising a CDR1 region of SEQ ID NO: 86, a CDR2 region of |
| | SEQ ID NO: 87 and a CDR3 region of SEQ ID NO: 81; |
| | An anti-TSLP antibody comprising a heavy chain variable domain |
| | comprising a CDR1 region of SEQ ID NO: 76, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 88 and a CDR3 region of SEQ ID NO: 81; |
| | An anti-TSLP antibody comprising a heavy chain variable domain |
| | comprising a CDR1 region of SEQ ID NO: 84, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 88 and a CDR3 region of SEQ ID NO: 81; or |
| | An anti-TSLP antibody comprising a heavy chain variable domain |
| | comprising a CDR1 region of SEQ ID NO: 76, a CDR2 region of SEQ ID |
| | NO: 85, and CDR3 region of SEQ ID NO: 78, and a light chain variable |
| | domain comprising a CDR1 region of SEQ ID NO: 86, a CDR2 region of |
| | SEQ ID NO: 80 and a CDR3 region of SEQ ID NO: 81. |
| | An anti-TSLP antibody comprising a heavy chain variable domain |
| | comprises SEQ ID NO: 89 and a light chain variable domain comprises |
| | SEQ ID NO: 90; |
| | An anti-TSLP antibody comprising a heavy chain variable domain |
| | comprises SEQ ID NO: 89 and a light chain variable domain comprises |
| | SEQ ID NO: 91; |
| | An anti-TSLP antibody comprising a heavy chain variable domain |
| | comprises SEQ ID NO: 92 and a light chain variable domain comprises |
| | SEQ ID NO: 93; |
| | An anti-TSLP antibody comprising a heavy chain variable domain |
| | comprises SEQ ID NO: 89 and a light chain variable domain comprises |
| | SEQ ID NO: 94, |
| U.S. Pat. No. | An anti-TSLP antibody comprising heavy chain variable region |
| 8,637,019 | comprising: a CDR-H1 sequence comprising SEQ ID NO: 95, a CDR-H2 |
| | sequence comprising SEQ ID NO: 96, and a CDR-H3 sequence |
| | comprising SEQ ID NO: 97; and/or an antibody light chain variable |
| | region or a TSLP-binding fragment thereof, said light chain variable |
| | region comprising: a CDR-L1 sequence comprising SEQ ID NO: 98, a |
| | CDR-L2 sequence comprising SEQ ID NO: 99, and a CDR-L3 sequence |
| | comprising SEQ ID NO: 100. |
| | An anti-TSLP antibody comprising a heavy chain variable region |
| | comprises the amino acid sequence of SEQ ID NO: 101 and the light |
| | chain variable region comprises the amino acid sequence of SEQ ID |
| | NO: 102. |
| | An anti-TSLP antibody comprising SEQ ID NO: 103 and SEQ ID |
| | NO: 104. |

Also contemplated are methods for treating chronic obstructive pulmonary disease (COPD) in a subject comprising administering an anti-TSLP antibody or antibody variant as described herein in in a dose of 280 mg to 420 mg at an interval of every 2 weeks or every 4 weeks.

It is contemplated that the subject to be treated is human. The subject may be an adult, an adolescent or a child.

Therapeutic antibody (or antibody variant) compositions may be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly, every 2 weeks, every 3 weeks, monthly, bimonthly, or at a longer interval.

In various embodiments, the amounts of therapeutic agent, such as a bivalent antibody having two TSLP binding sites, in a given dosage may vary according to the size of the individual to whom the therapy is being administered as well as the characteristics of the disorder being treated.

In exemplary treatments, the anti-TSLP antibody or antibody variant is administered in a dose range of about 280 mg to about 420 mg per dose. In various embodiments, the dose may be given in about 210 mg, 280 mg or 420 mg. In various embodiments, the anti-TSLP antibody or antibody variant may be administered at a dose of about 210, 220, 230, 240, 250, 260, 270, 280, 290, 300, 310, 320, 330, 340, 350, 360, 370, 380, 390, 400, 410 or 420 mg per dose. These concentrations may be administered as a single dosage form or as multiple doses. The above doses are given every two weeks or every four weeks. In various embodiments, the anti-TSLP antibody or antibody variant is administered at a single dose of 280 mg every two weeks or every four weeks. In various embodiments, the anti-TSLP antibody or antibody variant is administered at a single dose of 420 mg every two weeks or every four weeks. In various embodiments, the anti-TSLP antibody or antibody variant is administered at a single dose of 210 mg every two weeks or every four weeks.

For antibody variants, the amount of antibody variant should be such that the number of TSLP binding sites that are in the dose have an equimolar number of TSLP binding sites to canonical bivalent antibody described above.

It is contemplated that the anti-TSLP antibody or antibody variant is administered every 2 weeks or every 4 weeks for a period of at least 4 months, 6 months, 9 months, 1 year or more. In various embodiments, the administration is subcutaneous or intravenous.

Treatment with the anti-TSLP antibody or antibody variant is contemplated to improve one or more measures of atopic dermatitis including Investigator's Global Assessment (IGA) score, Eczema Area and Severity Index (EASI) score, Pruritus Numeric Rating Scale, Patient Oriented Eczema Measure, Dermatology Quality of Life Index (DLOI), EuroQOL quality of life 5-dimensions 3-level version (EQ-5D-3L)

In one embodiment, the administration improves one or more symptoms of atopic dermatitis including itching (pruritus), bleeding, oozing, cracked, flaking, and dry/rough skin, impact on sleep, erythema, induration/papulation, excoriation, and lichenification of skin, optionally as measured by atopic dermatitis patient electronic diary.

In various embodiments, the IGA score is reduced by one, two or three points. In various embodiments, the subject has an IGA score of 0 (clear) or 1 (almost clear) (IGA 0/1) at week 16 after treatment.

In various embodiments, the EASI is reduced by 10%, 15%, 25%, 30%, 35%, 40%, 45%, 50%, 55%, 60%, 65%, 70%, 75%, 80%, 85%, 90% or more. In various embodiments there is a 75% reduction in EASI (EASI 75) at week 16 after treatment. In various embodiments, EASI is reduced 20% to 70% at week 12 after treatment compared to baseline or placebo group, or EASI is reduced 50% to 90% (EASI 50/90) at week 16 after treatment compared to baseline or placebo group. In various embodiments, the time needed to reach EASI 50/75/90 is reduced in subjects receiving anti-TSLP antibody compared to subjects not receiving anti-TSLP.

In various embodiments, the SCORAD score of the subject is reduced by 20%, 25%, 30%, 35%, 40%, 45%, 50%, 55%, 60%, 65%, 70%, 75%, 80%, 85%, 90% or more compared to subjects not receiving anti-TSLP.

In various embodiments, treatment with anti-TSLP modulates the levels of one or more biomarkers of AD, including, cytokines, IgE, CCL17, CCL18, CCL22, and RNA transcriptional changes in blood and lesional versus nonlesional skin. In various embodiments, treatment with anti-TSLP reduces the level of Th2 cytokines. In various embodiments, the treatment modulates levels of or activity of IL-4, IL-5, IL-13, IL-17, IL-22, IL-23, IL-31, and/or IL-33, or combinations thereof.

The treatment also improves one or more symptoms of atopic dermatitis as measured by an atopic dermatitis symptom diary. Symptoms include itching and pruritus as measured by Pruritus Numeric Rating Scale; itching, bleeding, oozing, cracked, flaking, and dry/rough skin, and their impact on sleep erythema, induration/papulation, excoriation, and lichenification of skin as measured by Patient Oriented Eczema Measure or other patient reporting mechanism; dermatology-related symptoms and feelings measured by Dermatology Quality of Life Index (DLQI); mobility, self-care, usual activities, pain/discomfort, and anxiety/depression measured by the EuroQOL quality of life 5-dimensions 3-level version (EQ-5D-3L).

In various embodiments, treatment with the anti-TSLP antibody delays the time to an atopic dermatitis exacerbation or flare up compared to a subject not receiving the anti-TSLP antibody.

Also contemplated in the present disclosure is the administration of multiple agents, such as an antibody composition in conjunction with a second agent as described herein, including but not limited to an anti-inflammatory agent or atopic dermatitis therapy.

However, it is contemplated that, in various embodiments, the administration reduces frequency of or levels of co-administered therapy in the subject. Exemplary co-administered therapies include, but are not limited to, topical corticosteroids, topical calcineurin inhibitors, dupilumab, immunosuppressive or immunomodulating drugs (e.g., systemic corticosteroids, cyclosporine, mycophenolate-mofetil, interferon (IFN)-gamma, Janus kinase inhibitors, azathioprine, methotrexate), anti-IL-13 antibodies, anti-IL-5 pathway antibodies (benralizumab, mepolizumab, reslizumab), or combinations thereof. In various embodiments, the administration eliminates the need for corticosteroid therapy or other adjunct therapy.

### Formulations

In some embodiments, the disclosure contemplates use of pharmaceutical compositions comprising a therapeutically effective amount of an anti-TSLP antibody or antibody variant together with a pharmaceutically acceptable diluent, carrier, solubilizer, emulsifier, preservative, and/or adjuvant. In addition, the disclosure provides methods of treating a subject by administering such pharmaceutical composition.

In certain embodiments, acceptable formulation materials preferably are nontoxic to recipients at the dosages and concentrations employed. In certain embodiments, the pharmaceutical composition may contain formulation materials for modifying, maintaining or preserving, for example, the pH, osmolality, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

A suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor.

The formulation components are present preferably in concentrations that are acceptable to the site of administration. In certain embodiments, buffers are used to maintain the composition at physiological pH or at a slightly lower pH, typically within a pH range of from about 4.5 to about 8. Including about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, and about 8.0.

In various embodiments, the anti-TSLP antibody or antibody variant is in a formulation containing one or more basic amino acids (e.g., arginine, histidine or lysine) or salt thereof, or a calcium or magnesium salt, and a surfactant. In various embodiments, the formulation comprises 0.005% (w/v) to about 0.015% (w/v) polysorbate 20 or polysorbate 80. In various embodiments, the formulation is at pH between 4.5 and 6.8. In various embodiments, the antibody or antibody fragment in the formulation is at a concentration of greater than 140 mg/ml, e.g., from about 140 mg/ml to about 250 mg/ml, from about 160 mg/mL to about 250 mg/mL, or from about 140 mg/mL to about 210 mg/mL, or about 180 mg/ml or about 210 mg/ml. The formulation may be stored at 2° to 8° C. or −20° to −70° C. Exemplary formulations are described in co-owned applications U.S. 62/976,007 (attorney docket number 32053/54250P), U.S. 63/148,105 (attorney docket number 32053/54250P2) and PCT/US2021/17880.

When parenteral administration is contemplated, the therapeutic compositions for use may be provided in the form of a pyrogen-free, parenterally acceptable aqueous solution comprising the desired anti-TSLP antibody in a pharmaceutically acceptable vehicle. A particularly suitable vehicle for parenteral injection is sterile distilled water in which the antibody is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the antibody.

### EXAMPLES

### Example 1—A Phase 2 Study to Evaluate the Effect of Tezepelumab in Atopic Dermatitis

A limited phase 1 dose study of tezepelumab in the treatment of healthy subjects and atopic dermatitis patients showed that a single dose of tezepelumab had similar pharmacokinetics to healthy subjects, but effects on treatment of AD were inconclusive (Parnes et al., Clinical Pharmacology & Therapeutics 106:441-449, 2019). A phase 2a study (ALLEVIAD) demonstrated improvements in Investigator's Global Assessment (IGA) and Eczema Area and Severity Index (EASI) scoring, but did not meet statistical significance in the primary endpoint. (Simpson et al., J Am Acad Dermatol. 80:1013-1021, 2019).

In the ALLEVIAD study, not achieving statistical significance for the primary endpoint may have been due to the patient population selected, key study design elements surrounding topical corticosteroid (TCS) use, and length of treatment. In the ALLEVIAD study, a small number of enrolled patients (20%) had severe disease and most had moderate AD (as determined by IGA score). The current study requires a minimum of a 2-year history of AD and a higher EASI minimum score of 16, which may lead to a greater treatment effect and is consistent with other studies of biologic therapies for AD (Simpson et al, 2016).

In the ALLEVIAD study, placebo-treated patients applied greater TCS amounts during treatment versus tezepelumab-treated patients. In addition, all patients liberally applied high-strength TCS during the 2-week run-in period. These 2 factors potentially decreased the differential effect due to tezepelumab treatment. In the monotherapy design for Part A of the current study, subjects will wash out from TCS and are required to abstain from TCS use for 16 weeks. This design has been used for other therapies and will allow for an evaluation of efficacy with less potential confounding (Simpson et al, 2016).

Second, the 12-week treatment period in ALLEVIAD may not have been of sufficient duration to demonstrate efficacy in the AD population. Dosing was through week 10, thus, steady-state exposures to tezepelumab may not have been attained. In the current study, the longer treatment duration evaluation of the primary endpoint at week 16 will ensure that tezepelumab reaches steady-state concentrations.

Part A and Part B of this study consist of a 28-day screening period, a 52-week treatment period, and a 20-week safety follow-up period. During the 28-day screening period, subjects must discontinue from all topical AD therapies, except for approved moisturizers for at least the 7 consecutive days immediately prior to day 1.

Part A: Following the screening period, eligible subjects will be randomized 1:1:1:1 to receive 420 mg SC Q2W, 280 mg SC Q2W, 210 mg SC 04W, or placebo. All subjects receive a 420 mg SC dose (investigational product or placebo) as the first dose. Subjects will then receive the appropriate dose of investigational product at the week 2 visit, depending on treatment group to which they are randomized. Depending on if they are randomized to Q2W or 04W dosing, the dose at week 2 could be placebo or tezepelumab. Non-responders are defined as those subjects who have not achieved at least a 50% improvement in EASI at week 16 compared to baseline (day 1). Subjects who are determined to be non-responders in Part A will receive tezepelumab 420 mg SC Q2W for the remainder of the study, beginning with the week 18 dose.

These dose regimens will allow establishing exposure-response to aid in dose selection for future studies. A first dose of 420 mg will allow faster achievement of steady state for the lower dose regimens (210 mg SC Q4W and 280 mg SC Q2W), and is also anticipated to improve symptoms of AD (e.g., sleep, skin lesions, and pruritus).

Part B: Part B is a randomized, placebo-controlled, double-blind study designed to evaluate the safety and efficacy of tezepelumab when administered with moderate class TCS in adults with moderate-to-severe AD. Part B investigates the clinical response of tezepelumab 420 mg SC Q2W with TCS as adjunctive treatment. Inclusion of this cohort allows for an evaluation of the effects of tezepelumab in a more real-life scenario. While TCS use was permitted in the ALLEVIAD study, the criteria for TCS use in Part B of the current study has been modified to provide guidance to the investigator for when and how to use TCS. Following the screening period, eligible subjects are randomized 2:1 to receive 420 mg SC Q2W or placebo; both groups in Part B are allowed to use TCS after the day 1 visit. Non-responders in Part B will not be switched to 420 mg SC Q2W at week 18 and will remain on their initial dose for the duration of the study.

Subjects who use TCS are to apply medium potency TCS once daily to areas with active lesions. Low potency TCS should be used once daily on areas of thin skin (eg, face, neck, intertriginous, genital areas, areas of skin atrophy) or for areas where continued treatment with medium potency TCS is considered unsafe. Triamcinolone acetonide 0.1% cream or fluocinolone acetonide 0.025% ointment are recommended as medium potency TCS therapy; hydrocortisone 1c)/0 cream is recommended as low potency TCS therapy. After lesions are under control (clear or almost clear), subjects are to switch from medium potency to low potency TCS and treat once daily for 7 days, then cease TCS therapy.

If the lesions return, subjects are to resume treatment with medium potency TCS, and are to use the step-down approach to low potency TCS described above. For lesions that persist or worsen with once daily application of medium potency TCS, subjects may be treated (rescued) with high or super-high potency TCS, unless higher potency TCS is considered unsafe.

Subjects

Approximately 240 subjects will be enrolled in Part A of the study. Approximately 60 subjects will be enrolled in Part B of the study. A safety follow-up occurs for 20 weeks after the final dose of study drug (18 weeks after the end of treatment (EOT) visit) in Part A and Part B.

Subjects are eligible to be included in the study only if all of the following criteria apply:
Subject has provided informed consent prior to initiation of any study specific activities/procedures;
Age to 75 years at screening;
Clinical diagnosis of chronic AD (also known as atopic eczema) for at least 2 years prior to screening and has confirmed AD (Hanifin and Rajka criteria for AD; Hanifin and Rajka, 1980);
AD that affects 10% body surface area as assessed by EASI at screening and on day 1;
An IGA score of at screening and on day 1;
An EASI score of 16 at screening and on day 1;
Subject discontinued treatment with TCS, topical calcineurin inhibitors (TCI), and prescription moisturizers containing TCS or TCI for at least the 7 days immediately prior to the first dose of investigational product;
Documented recent history (within 12 months before the screening visit) of inadequate response to treatment with topical TCS or subjects for whom topical treatments are otherwise medically inadvisable (i.e., because of important side effects or safety risks).

An inadequate response is defined as failure to achieve and maintain remission or a low disease activity state (comparable to IGA 0=clear to IGA 2=mild) despite treatment with a daily regimen of TCS of medium or higher potency (with or without TCI as appropriate).

Subjects are excluded from the study if any of the following criteria apply:
Active dermatologic conditions, which might confound the diagnosis of AD or would interfere with the assessment of treatment, such as scabies, seborrheic dermatitis, cutaneous lymphoma, ichthyosis, psoriasis, allergic contact dermatitis, or irritant contact dermatitis;
History of a clinically significant infection within 28 days prior to day 1 that, in the opinion of the investigator or medical monitor, might compromise the safety of the subject in the study, interfere with evaluation of the investigational product, or reduce the subject's ability to participate in the study. Clinically significant infections are defined as either of the following:—1) a systemic infection; or—2) a serious skin infection requiring parenteral antibiotic, antiviral, or antifungal medication;
Diagnosis of a helminth parasitic infection within 6 months prior to screening that had not been treated with or had failed to respond to standard of care therapy;
Documented medical history of chronic alcohol or drug abuse within 12 months prior to screening;
History of anaphylaxis following any biologic therapy;
Evidence of active liver disease at screening, including jaundice or aspartate aminotransferase (AST), alanine aminotransferase (ALT), or alkaline phosphatase greater than twice the upper limit of normal (ULN);
Subjects who, in the opinion of the investigator, have evidence of active tuberculosis (TB), either treated or untreated, or a positive QuantiFERON-tuberculosis Gold (QFT-G) test for TB during screening;
Positive hepatitis B surface antigen or hepatitis C antibody serology. Subjects with a history of hepatitis B vaccination without a history of hepatitis B are allowed to enroll in the study;
Positive human immunodeficiency virus (HIV) test at screening or the subject is taking antiretroviral medications, as determined by medical history, prior medications, and/or the subject's verbal report.

Endpoints and Assessments

Primary Endpoints: The effect of tezepelumab treatment compared with placebo is assessed using the Investigator's Global Assessment (IGA) and Eczema Area and Severity Index (EASI).

Investigator's Global Assessment: The IGA uses clinical characteristics of erythema, infiltration, papulation, oozing, and crusting as guidelines for the overall severity assessment (Breuer et al, 2004). The IGA allows investigators to assess overall disease severity at one given time point and consists of a 5-point severity scale from clear to severe disease (0=clear; 1=almost clear; 2=mild disease; 3=moderate disease; 4=severe disease; 5=very severe disease). See the table below.

| IGA | |
|---|---|
| Score | Definition |
| 0 | Clear, no inflammatory signs of AD |
| 1 | Almost clear (just perceptible erythema, |
| | and just perceptible |
| | papulation/infiltration) |
| 2 | Mild Disease (mild erythema, and mild |
| | papulation/infiltration) |
| 3 | Moderate disease (moderate erythema, |
| | and moderate papulation/infiltration) |
| 4 | Severe disease (severe erythema, and |
| | severe papulation/infiltration) |
| 5 | Very severe disease (severe erythema, |
| | and severe papulation/infiltration with |
| | oozing/crusting) |

Eczema Area and Severity Index. The EASI was designed by modifying the Psoriasis Area and Severity Index, which has been widely used in clinical trials and has been established as a well-accepted and standardized instrument for assessing therapeutic response in patients with psoriasis (Schmitt et al, 2007). The EASI evaluates 4 natural anatomical regions for severity and extent of key disease signs and focuses on key acute and chronic signs of inflammation (i.e., erythema, induration/papulation, excoriation, and lichenification). The maximum score is 72, with higher values indicating more severe disease.

Endpoints include an IGA score of 0 (clear) or 1 (almost clear) (IGA 0/1) at week 16 post-treatment and a 75% reduction in EASI (EASI 75) at week 16 post-treatment. A positive treatment effect is a difference in response rates (tezepelumab minus placebo) of 27% for IGA 0/1 and 35% for EASI 75, under the assumption of placebo rates of 10% for IGA 0/1 and 15% for EASI 75. The treatment effect thresholds of 27% and 35% may be adjusted if the observed placebo rates are different from the assumptions above.

Secondary Endpoints: Secondary endpoints include, estimating the effect of tezepelumab compared with placebo on the efficacy measure of EASI secondary endpoint include 50% and 90% reduction in EASI (EASI 50/90) at week 16; estimating the time needed to reach EASI 50/75/90, e.g., time at which EASI 50/75/90 is achieved from day 1; estimating the effect of tezepelumab compared with placebo on the efficacy measure of Scoring Atopic Dermatitis (SCORAD) at week 16 post-treatment; estimating the effect of tezepelumab compared with placebo on the patient reported outcome (PRO) measure of pruritus assessed using a numeric rating scale (NRS) (Pruritus NRS) at week 16 post-treatment; and characterizing the pharmacokinetics (PK) of tezepelumab as assessed by serum trough concentrations of tezepelumab at scheduled visits.

Additional outcomes assessments include estimating the long-term effect of tezepelumab on the efficacy measures of EASI, SCORAD, and Pruritus NRS, e.g., IGA 0/1 at week 24, week 36, and week 52, EASI 75 at week 24, week 36, and week 52, EASI 50/90 at week 24, week 36, and week 52, SCORAD at week 24, week 36, and week 52, Pruritus NRS at week 24, week 36, and week 52; investigating potential biomarker development by biochemical analysis of blood or skin samples, which may include but are not limited to, IgE, CCL17, CCL22, and RNA transcriptional changes in blood and lesional versus nonlesional skin; determining the effect of tezepelumab on Patient Global Impression of Severity (PGI-S), e.g., change from baseline in PGI-S at scheduled visits; evaluating the effect of tezepelumab on health-related quality of life (HRQoL) assessed using the Dermatology Life Quality Index (DLQI), e.g., Change from baseline in DLQI at scheduled visits; evaluate the immunogenicity of tezepelumab, e.g., as measured by anti-tezepelumab antibodies; and assess the effects of drug target genes and AD genes and/or subject response to tezepelumab, e.g., by assessment of Gene polymorphisms that may influence clinical response to study drug, such as filaggrin and TSLP.

Further outcome measures are taken for Part B, including evaluating the effect of tezepelumab compared with placebo, assessed using the IGA and EASI, e.g., IGA score of 0 (clear) or 1 (almost clear) (IGA 0/1) at week 16 and EASI 75 at week 16; estimating the effect of tezepelumab compared with placebo on the efficacy measure of EASI. EASI 50/90 at week 16; estimating the time needed to reach EASI 50/75/90, e.g., time at which EASI 50/75/90 is achieved from day 1; estimating the effect of tezepelumab compared with placebo on the efficacy measure of SCORAD at week 16; estimating the effect of tezepelumab compared with placebo on the PRO measure of pruritus assessed using an NRS (Pruritus NRS) at week 16 post-treamtne; characterizing the PK of tezepelumab e.g., using serum trough concentrations of tezepelumab at scheduled visits; and establishing the safety and tolerability of tezepelumab compared with placebo via analysis of subject incidence of adverse events.

Scoring of Atopic Dermatitis. The SCORAD is a clinical tool for assessing the severity (i.e., extent, intensity) of AD. The tool evaluates the extent and intensity of the AD lesions, along with subjective symptoms (Kunz et al, 1997). The maximum total score is 103, with higher values indicating more severe disease.

Patient-reported Outcomes (PRO). The electronic diary (eDiary), which includes the pruritus NRS, will be completed by the subject at home each morning during the treatment and follow-up periods.

Electronic Diary. The electronic diary includes one item to capture subject-reported pruritus. The diary will be completed by the subject at home each morning using an electronic device according to the Schedule of Activities.

Pruritus Numeric Rating Scale. Pruritus will be assessed using an numeric rating scale (NRS) (0-10) with 0=no itch and 10=worst imaginable itch. Subjects complete the NRS as part of the daily diary each day in the morning.

Patient Oriented Eczema Measure. The Patient Oriented Eczema Measure (POEM) is a 7-item validated questionnaire used in clinical practice and clinical trials to assess disease symptoms in children and adults (Charman, 2004). It evaluates time spent in the past week with AD signs and symptoms: individual items of itching, bleeding, oozing, cracked, flaking, and dry/rough skin, and their impact on sleep.

Dermatology Quality of Life Index. The DLQI is a 10-item, subject-completed, HRQoL assessment with content specific to those with dermatology conditions. The recall period is 1 week (Finlay and Kahn, 1994). The DLQI content captures respondent perceptions of dermatology-related symptoms and feelings (embracement), impacts on daily activities, leisure, work or school, personal relationships, and the side effects of treatment. Each item is scored on a 4-point Likert scale: 0=not at all/not relevant; 1=a little; 2=a lot; and 3=very much (Basra et al, 2008).

EQ-5D-3L. The EuroQOL quality of life 5-dimensions 3-level version (EQ-5D-3L) is a standardized instrument for use as a measure of health-related quality of life (HRQoL) and was developed by EuroQol (Brooks, 1996). It defines health in terms of 5 dimensions: mobility, self-care, usual activities, pain/discomfort, and anxiety/depression. Each dimension has 3 ordinal levels of severity: 1, no problem; 2, some problems; and 3, severe problems. Overall health state is defined as a 5-digit number.

Patient Global Impression of Severity. The PGI-S is a single item designed to capture the subject's perception of overall symptom severity at the time of completion on a 5-point categorical response scale (no symptoms to very severe symptoms).

Adverse Events: All adverse events observed by the investigator or reported by the subject that occur after the first dose of investigational product through the end of study/safety follow-up visit or 20 weeks after the last administration of investigational product are to be collected/reported.

Biomarkers

Serum Immunoglobulins. Approximately 80% of AD patients have extrinsic AD (defined as increased IgE levels [>150 kU/L]) and increased allergen reactivity (Novak and Bieber, 2003). Conversely, approximately 20% of AD patients present the same dermatological disease, but have normal levels of serum IgE and are not allergen reactive (i.e., intrinsic AD). Serum samples will be collected from all subjects to determine extrinsic vs intrinsic disease based on baseline (day 1) total serum IgE levels. To assess the impact of tezepelumab on total Ig levels, serum IgE/IgA/IgG/IgM levels are measured.

Staphylococcus Aureus Characterization. Staphylococcus aureus (S. aureus) has the potential to exacerbate AD skin disease through colonization, super-infection of the skin, and production of inflammation-inducing toxins. Cotton swab samples are collected from nonlesional and lesional skin to characterize S. aureus colonization and infection to determine whether differential benefit is observed in S. aureus-positive or—negative subjects.

Safety Biomarkers (Serum Tryptase). Blood samples are collected at day 1 for baseline assessment of safety biomarkers (serum tryptase). If a suspected anaphylactic reaction occurs during or within a 24-hour period after administration of investigational product, an additional sample of whole blood for assessment of safety biomarkers will be collected as soon as possible after the event, at 60 minutes ±30 minutes after the event, and at discharge from the study center.

Pharmacokinetic Assessments: Whole blood samples of approximately 5 mL are collected for measurement of serum concentrations of tezepelumab.

Pharmacodynamic Assessments. Venous blood samples are collected from all subjects at the time points outlined to determine the effect of tezepelumab on the levels of circulating CCL17 (TARC) and CCL22 (MDC), which are systemically elevated in subjects with AD (Hijnen et al, 2004; Shimada et al, 2004) and downstream of TSLP signaling (Soumelis et al, 2002).

Pharmacogenetic Assessments. DNA analyses may be performed. These optional pharmacogenetic analyses focus on inherited genetic variations to evaluate their possible correlation to the disease and/or responsiveness to the therapies used in this study. The goals of the optional studies include the use of genetic markers to help in the investigation of moderate-to-severe AD and/or to identify subjects who may have positive or negative response to investigational product or protocol-required therapies.

Antibody Testing Procedures: Bioanalytical testing for anti-tezepelumab antibodies may be conducted if there are unexpected PK findings or safety-related concerns in the study population that warrant further investigation. Samples that test positive for anti-tezepelumab antibodies may be further characterized.

Blood Biomarkers: Blood samples (serum/plasma and whole blood) are collected as directed for the assessment of changes in circulating biomarker levels that are elevated in subjects with AD. Additional assays which may be performed on blood samples include measuring changes in mRNA transcripts after tezepelumab treatment.

Skin RNA Transcript Profiling: Skin punch biopsy samples will be collected from a subset of consenting subjects as specified for RNA transcripts. The purpose of these analyses will be to retrospectively evaluate transcript biomarkers predictive of subject response at baseline, prior to investigational product administration, as well as to potentially identify additional AD biomarkers.

Statistical Analysis

For the primary endpoints of IGA 0/1 and EASI 75 at week 16, the treatment effect will be tested using a logistic regression model adjusting for covariates. From this model, odd ratios and 95% Cls will be reported comparing each tezepelumab dose group to placebo. In addition, the percentage of subjects in each treatment group with a response and the difference in the percentage of subjects responding between each tezepelumab dose group and placebo will be summarized with a 95% confidence interval.

In Part A of this study, a total of 240 subjects are randomized in a 1:1:1:1 ratio to tezepelumab 420 mg SC Q2W, 280 mg SC Q2W, 210 mg SC 04W, and placebo (approximately 60 in each group). There is at least 85% power for each co-primary endpoint, assuming IGA 0/1 response rates of 10% and 40% for placebo and the highest tezepelumab dose group (420 mg SC Q2W), respectively, and assuming EASI response rates of 15% and 50%, respectively. These assumed treatment effects are based on the results of other new agents in this indication (DUPIXENT, 2017). These calculations used a 2-sidedX₂ test at a significance level of 0.025 (to reflect the Bonferonni-based gatekeeping procedure) and assumed a 10% dropout rate.

Results:

A total of 251 subjects were randomized (63, 63, 62 and 63 in teze 420 mg/Q2W, 280 mg/Q2W, 210 mg/Q4W or placebo, respectively). 250 subjects received at least one dose of investigational product. 75 (30.0%) subjects received rescue medication of topical corticosteroid or topical calcineurin inhibitors, similar proportions were observed among treatment arms. A total of 134 (53.4%) subjects were EASI 50 non-responders at week 16, where 130 subjects were switched to 420 Q2W after week 16:40 (63.5%)— Placebo, 38 (60.3%)—420 mg/Q2W, 30 (47.6%)—280 mg/Q2W, 26 (41.9%)— 210 mg/Q4W.

Analysis of co-primary endpoints of IGA 0/1 and EASI 75 at week 16 did not result in statistically significant changes between treatment and placebo groups. Non-statistically-significant treatment effects were observed for the 420 mg/Q2W and 280 mg/Q2W groups on relevant secondary endpoints at week 16. See Table 1.

TABLE 1

| | | Tezepelumab | | |
|---|---|---|---|---|
| | Placebo | 210 mg Q4W | 280 mg Q2W | 420 mg Q2W |
| IGA 0/1 at week 16 | (N = 63) | (N = 62) | (N = 63) | (N = 63) |
| IGA 0/1 - n^{a} (%) | 2 (3.2) | 4 (6.5) | 2 (3.2) | 5 (7.9) |
| Odds ratio^{b} (95% CI) | | 1.686 (0.290, | 1.011 (0.135, | 2.146 (0.390, |
| | | 9.809) | 7.551) | 11.800) |
| p-value | | 0.56 | 0.99 | 0.38 |

N = Number of subjects in the Full Analysis Set - Part A
Subjects who received rescue medication of TCS/TCI from day 29 to week 16 are considered non-responders.
CI = Confidence Interval; IGA = Investigator's Global Assessment
Missing data was imputed using non-responder imputation.
^{a} Number of subjects whose IGA score is 0 or 1 at week 16 and who did not receive rescue medication of TCS/TCI from day 29 to week 16.
^{b} The odds ratio is obtained from a logistic regression model with baseline IGA as covariate.

IGA 0/1 at week 16 was not statistically different from placebo in 420 mg/Q2W, 280 mg/Q2W and 210 mg/Q4W groups with odd ratio of 2.15 (95% CI: 0.39, 11.80, nominal p-value=0.38), 1.01 (95′)/oCI: 0.14, 7.55, nominal p-value=0.99), and 1.67 (95% CI: 0.29, 9.81, nominal p-value=0.56), respectively. Overall response rates for all groups were low (less than 12%).

EASI 75 at week 16 was not statistically different from placebo in the 420 mg/Q2W, 280 mg/Q2W, and 210 mg/Q4W groups, with odds ratio of 0.73 (95% CI: 0.24, 2.20, nominal p-value=0.58), 1.22 (95% CI: 0.44, 3.36, nominal p-value=0.70) and 0.98 (95% CI: 0.34, 2.80, nominal p-value=0.97), respectively. See Table 2.

TABLE 2

| | | Tezepelumab | | |
|---|---|---|---|---|
| | Placebo | 210 mg Q4W | 280 mg Q2W | 420 mg Q2W |
| EASI 75 at week 16 | (N = 63) | (N = 62) | (N = 63) | (N = 63) |
| EASI 75 - n^{a} (%) | 8 (12.7) | 9 (14.5) | 10 (15.9) | 7 (11.1) |
| Odds ratio^{b} (95% CI) | | 0.982 (0.344, | 1.217 (0.441, | 0.730 (0.243, |
| | | 2.803) | 3.356) | 2.197) |
| p-value | | 0.97 | 0.70 | 0.58 |

N = Number of subjects in the Full Analysis Set - Part A
Subjects who received rescue medication of TCS/TCI from day 29 to week 16 are considered non-responders.
CI = Confidence Interval; EASI = Eczema Area and Severity Index
Missing data was imputed using non-responder imputation.
^{a} Number of subjects with greater than or equal to 75% improvement in EASI score from baseline and who did not receive rescue medication of TCS/TCI from day 29 to week 16.
^{b}The odds ratio is obtained from a logistic regression model with baseline EASI covariate.

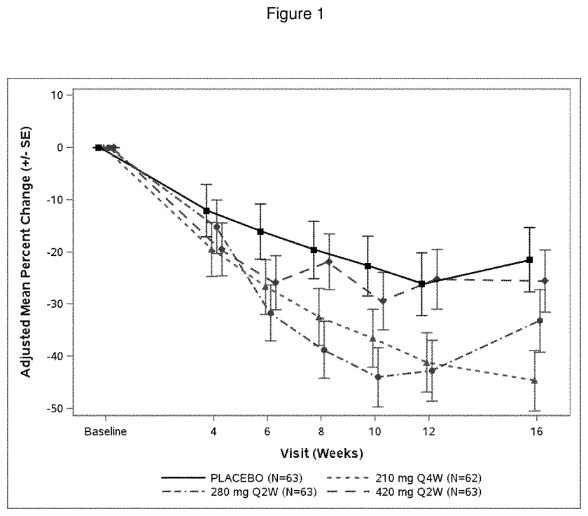

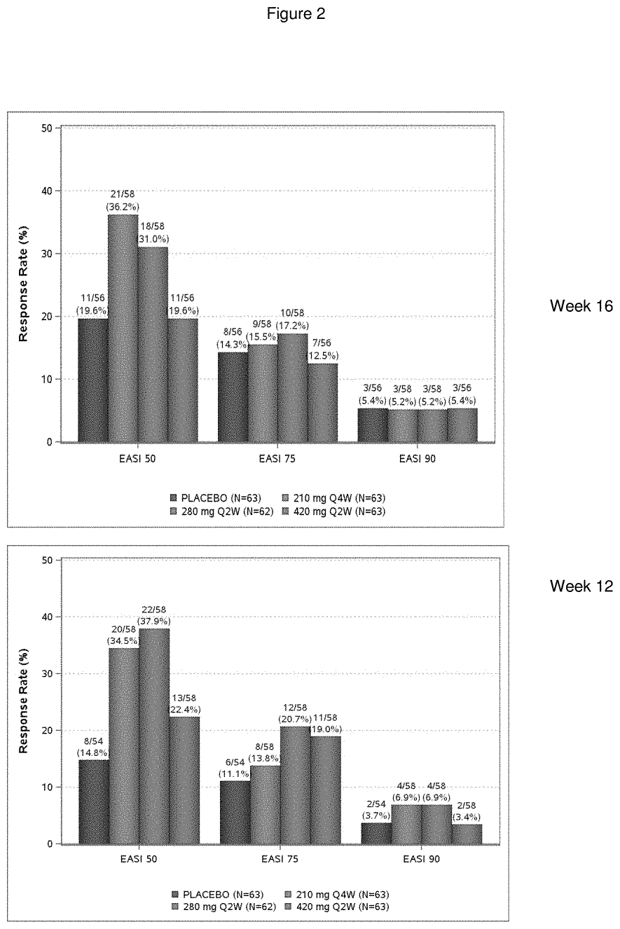

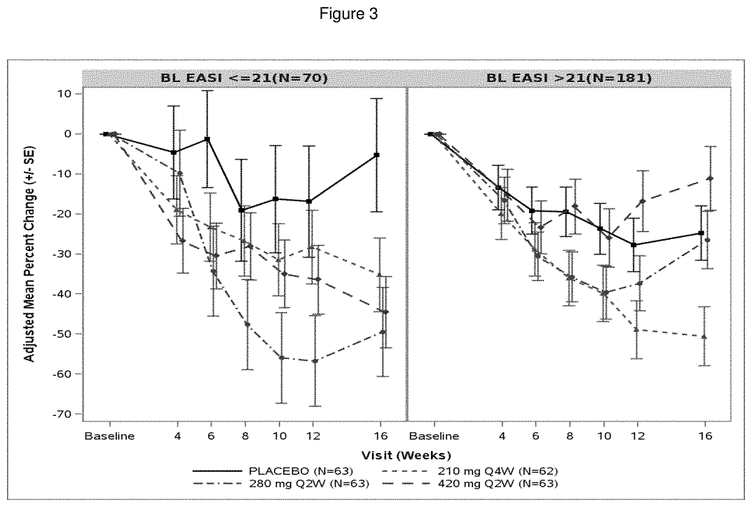

Overall EASI 75 response rates for all dose groups at week 16 were low (<23%) and no significant treatment differences among dose groups and placebo were detected. Results are depicted in FIG. 1. At week 12 and earlier timepoints, the 280 mg/Q2W treatment group demonstrated improvements on several efficacy measures, including EASI 75 and EASI % change from baseline. However, the 210 mg/Q4W treatment group demonstrated improvements compared to placebo at week 16 in the following continuous measures: EASI percent change from baseline: LS mean difference=−23.1% (p=0.006) (FIG. 1); EASI change from baseline: LS mean difference=−6.88 (p=0.005); IGA change from baseline: LS mean difference=−0.49 (p=0.007); SCORAD (Scoring Atopic Dermatitis) change from baseline: LS mean difference=−9.24 (p=0.009). Comparison between week 16 and week 12 is shown in FIG. 2. Additionally, a slightly better efficacy response rate was observed in moderate AD (BL EASI 21) relative to severe AD (FIG. 3).

It was also noted that Subjects in 210 mg/Q4W and 420 mg/Q2W groups were less severe. The proportion of subjects with higher Investigator Global Assessment (IGA 4) was slightly lower in 210/mg/04W (40.3%) and 420 mg/Q2W (41.3%) groups compared to those in placebo and 280 mg/Q2W (58.7% each). The mean Eczema Area and Severity Index (EASI) score in the placebo group (mean=32.0) was slightly higher than in the other treatment groups (mean=28.4, 30.3 and 28.6 for 210 mg/Q4W, 280 mg/Q2W and 420 m/Q2W respectively). Mean level of eosinophil and Serum IgE were lower in the 210 mg/Q4W group than in the other groups.

PK Model-predicted concentrations are consistent with observed concentrations. Higher exposures were observed for subjects receiving higher dose/dosing frequency. Steady-state was attained by Week 12 for 280 mg/Q2W (+LD) and 420 mg/Q2W and by Week 4 for 210 mg/Q4W (+LD).

No unusual treatment related adverse events were reported.

Numerous modifications and variations of the invention as set forth in the above illustrative examples are expected to occur to those skilled in the art. Consequently only such limitations as appear in the appended claims should be placed on the invention.

### REFERENCES

Basra M K, Fenech R, Gatt R M, et al. The Dermatology Life Quality Index 1994-2007: a comprehensive review of validation data and clinical results. Br J Dermatol. 2008; 159(5):997-1035.
Beck L A, Leung D Y. Allergen sensitization through the skin induces systemic allergic responses. J Allergy Clin Immunol. 2000; 106(5 suppl):5258-63.
Bieber T, Leung D Y, editors. Atopic dermatitis. New York: Marcel Dekker, 2002.
Breuer K, Braeutigan M, Kapp A, et al. Influence of pimecrolimus cream 1% on different morphological signs of eczema in infants with atopic dermatitis. Dermatology. 2004; 209(4):314-320.
Brooks R. EuroQol: the current state of play. Health Policy. 1996; 37(1): 53-72.
Charman C R, Venn A J, Williams H C. The patient-oriented eczema measure: development and initial validation of a new tool for measuring atopic eczema severity from the patients' perspective. Arch Dermatology. 2004; 140(12):1513-1519.
Chi C C, Kirtschig G, Aberer W, et al. Evidence-based (S3) guideline on topical corticosteroids in pregnancy. Br J Dermatol. 2011; 165:943-952.
Demehri S, Morimoto M, Holtzman M J, Kopan R. Skin-derived TSLP trigger progression from epidermal-barrier defects to asthma. PLos Biol. 2009; 19;7(5):e1000067.
DUPIXENT (Dupilumab) [instructions for use]. Tarrytown, N.Y.: Regeneron Pharmaceuticals, Inc. and Sanofi-Aventis US, LLC; 2017.
Ebner S, Nguyen V A, Forstner, M, Wang Y H, Wolfram D, Liu Y J, et al. Thymic Stromal Lymphopoietin converts human epidermal Langerhans cells into antigen-presenting cells that induce proallergic T cells. J Allergy Clin Immunol. 2007; 119:982-990.
Finlay A Y, Khan G K. Dermatology Life Quality Index (DLQI)— a simple practical measure for routine clinical use. Clin Exp Dermatol. 1994; 19(3):210-216.
Hanifin J M, Reed M L. Eczema Prevalence and Impact Working Group. A population-based survey of eczema prevalence in the United States. Dermatitis. 2007; 18(2):82-91.
Hanifin J M, Rajka G. Diagnostic features of atopic dermatitis. Acta Derm Venereol Suppl (Stockh). 1980; 92:44-47.
He R, Oyoshi M K, Garibyan L, Kumar L, Ziegler S F, Geha R S. TSLP acts on infiltrating effector T cells to drive allergic skin inflammation. Proc Natl Acad Sci. USU 2008; 105; 33:11875-11880.
Hijnen D, De Bruin-Weller M, Oosting B, et al. Serum thymus and activation-regulated chemokine (TARO) and cutaneous T cell-attracting chemokine (CTACK) levels in allergic diseases: TARO and CTACK are disease-specific markers for atopic dermatitis. J Allergy Clin Immunol. 2004; 113(2):334-340.
Kiebert G, Sorensen S V, Revicki D, Fagan S C, Doyle J J, Cohen J, et al. Atopic dermatitis is associated with a decrement in health-related quality of life. Int J Dermatol. 2002; 41(3):151-158.
Kim J, Kim B E, Lee J, et al. Epidermal thymic stromal lymphopoietin predicts the development of atopic dermatitis during infancy. J Allergy Clin Immunol. 2016; 137(4):1282-1285.
Kunz B, Oranie A P, Labreze L et al. Clinical validation and guidelines for the SCORAD index: consensus report of the European Task Force on Atopic Dermatitis. Dermatology. 1997; 195(1):10-19.
Novak N, Bieber T. Allergic and nonallergic forms of atopic diseases. J Allergy Clin Immunol. 2003; 112(2):252-262.
Oh M H, Oh S Y, Yu J, Myers AC, Leonard W J, Liu YJ et al. IL-13 induces skin fibrosis in atopic dermatitis by thymic stromal lymphopoietin. J Immunol. 2011; 186:7232-7242.
Ring J, Alomar A, Bieber T, Deleuran M, Fink-Wagner A, Gelmetti C, et al; European Dermatology Forum (EDF); European Academy of Dermatology and Venereology (EADV); European Federation of Allergy (EFA); European Task Force on Atopic Dermatitis (ETFAD); European Society of Pediatric Dermatology (ESPD); Global Allergy and Asthma European Network (GA2LEN). Guidelines for treatment of atopic eczema (atopic dermatitis) part I. J Eur Acad Dermatol Venereol. 2012; 26(8):1045-1060.
Schmitt J, Langan S, Williams H C; European Dermato-Epidemiology Network. What are the Best Outcome Measurements for Atopic Eczema? A Systematic Review. J Allergy Clin Immunol. 2007; 120:1389-1398.
Shimada Y, Takehara K, Sato S. Both Th2 and Th1 chemokines (TARC/CCL17, MDC/CCL22, and Mig/CKCL9) are elevated in sera from patients with atopic dermatitis. J Dermatol Sci. 2004; 34(3):201-208.
Silverberg J I, Hanifin J M. Adult eczema prevalence and associations with asthma and other health and demographic factors: A US population-based study. J Allergy Clin Immunol. 2013; 132:1132-1138.
Silverberg J I, Silverberg N B, Lee-Wong M. Association between atopic dermatitis and obesity in adulthood. Br J Dermatol. 2012; 166(3): 498-504.
Simpson E L, Beiber T, Guttman-Yassky L A, Beck A, Blauvelt M J, Cork J I, et al. Two phase 3 trials of Dupilumab versus placebo in atopic dermatitis. N Engl J Med. 2016; 375(24):2335-2348.
Soumelis V, Reche P A, Kanzler H, Yuan W, Edward G, Homey B, et al. Human epithelial cells trigger dendritic cell mediated allergic inflammation by producing TSLP. Nat Immunol. 2002; 3(7):673-680.
Spergel J M, AS Paller. Atopic dermatitis and the atopic march. J Allergy Clin Immunol. 2003; 112(6 suppl):S118-S127. Tezepelumab Investigator's Brochure. Thousand Oaks, Calif. Amgen nc.
Yoo J, Omori M, Gyarmati D, Zhou B, Aye T, Brewer A, et al. Spontaneous atopic dermatitis in mice expressing an inducible thymic stromal lymphopoietin transgene specifically in the skin. J Exp Med. 2005; 4:541-549.
Zhang Z, Hener P, Frossard N, Kato S, Metzger D, Li M, et al. Thymic stromal lymphopoietin overproduced by keratinocytes in mouse skin aggravates experimental asthma. PNAS. 2009; 106(5):1536-1541.
Zheng T, Yu J, Oh M H, Zhu Z. The Atopic March: Progression from Atopic Dermatitis to Allergic Rhinitis and Asthma. Allergy Asthma Immunol Res. 2011; 3(2):67-73.

What is claimed:

1. A method for treating atopic dermatitis in a subject comprising administering a therapeutically effective amount of an anti-TSLP antibody or antibody variant in a dose of 280 mg to 420 mg at an interval of every 2 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

2. A method for treating atopic dermatitis in a subject comprising administering a therapeutically effective amount of an anti-TSLP antibody or antibody variant in a dose of 280 mg to 420 mg at an interval of every two weeks, wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

3. The method of claim 1 or 2, wherein the antibody or antibody variant is administered every 4 weeks.

4. The method of any one of claims 1 to 3, wherein the antibody is an IgG2 antibody.

5. The method of any one of claims 1 to 4, wherein the antibody or antibody variant is administered at a dose of 280 mg.

6. The method of any one of claims 1 to 4, wherein the antibody or antibody variant is administered at a dose of 420 mg.

7. A method for treating atopic dermatitis in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2.

8. A method for treating atopic dermatitis in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b), wherein the antibody specifically binds to a TSLP polypeptide as set forth in amino acids 29-159 of SEQ ID NO:2.

9. The method of any one of the preceding claims, wherein the antibody or antibody variant is administered for a period of at least 4 months, 6 months, 9 months, 1 year or more.

10. The method of any one of the preceding claims, wherein said anti-TSLP antibody or antibody variant thereof is bivalent and selected from the group consisting of a human antibody, a humanized antibody, a chimeric antibody, a monoclonal antibody, a recombinant antibody, an IgG1 antibody, an IgG2 antibody, an IgG3 antibody, and an IgG4 antibody.

11. The method of any one of claims 7-10, wherein the antibody is an IgG2 antibody.

12. The method of any one of the preceding claims, wherein the antibody or antibody variant is a human antibody.

13. The method of any one of the preceding claims wherein, the antibody or antibody variant further comprises a pharmaceutically acceptable carrier or excipient.

14. The method of any one of the preceding claims, wherein the atopic dermatitis is severe or moderate atopic dermatitis.

15. The method of any one of the preceding claims, wherein the atopic dermatitis is lesional or non-lesional atopic dermatitis.

16. The method of any one of the preceding claims, wherein the atopic dermatitis is chronic or acute atopic dermatitis.

17. The method of any one of the preceding claims, wherein the atopic dermatitis is intrinsic or extrinsic dermatitis.

18. The method of any one of the preceding claims, wherein the subject is an adult.

19. The method any one of the preceding claims, wherein the subject is a child or adolescent.

20. The method any one of the preceding claims, wherein the administration improves one or more measures of atopic dermatitis in a subject selected from the group consisting Investigator's Global Assessment (IGA) score, Eczema Area and Severity Index (EASI) score, Pruritus Numeric Rating Scale, Patient Oriented Eczema Measure, Dermatology Quality of Life Index (DLQI), EuroQOL quality of life 5-dimensions 3-level version (EQ-5D-3L).

21. The method any one of the preceding claims, wherein the administration improves one or more symptoms of atopic dermatitis as measured by a patient symptom diary.

22. The method of any one of the preceding claims, wherein the administration improves one or more symptoms of atopic dermatitis selected form the group consisting of itching (pruritus), bleeding, oozing, cracked, flaking, and dry/rough skin, impact on sleep, erythema, induration/papulation, excoriation, and lichenification of skin.

23. A method for treating atopic dermatitis in a subject comprising administering a therapeutically effective amount of an anti-TSLP antibody or antibody variant in a dose of 280 to 420 mg at an interval of every 2 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antibody specifically binds to a TSLP polypeptide as set forth in amino acids 29-159 of SEQ ID NO:2, wherein the antibody is an IgG2 antibody.

24. The method of claim 23, wherein the antibody is administered every 4 weeks.

25. The method of claim 23 or 24, wherein the antibody is administered at a dose of 280 mg.

26. The method of claim 23 or 24, wherein the antibody is administered at a dose of 420 mg.

27. The method of any one of the preceding claims, wherein the antibody is tezepelumab.

28. The method of claim 27, wherein the antibody is an IgG2 antibody, and has the full length heavy and light chain sequences set out in SEQ ID NOs: 105 and 106, respectively.

29. The method of any one of the preceding claims, wherein the antibody variant has substantially similar pK characteristics as tezepelumab in humans.

30. A method of reducing the frequency of atopic dermatitis exacerbation in a subject comprising administering a therapeutically effective amount of an anti-TSLP antibody or antibody variant in a dose of 280 mg to 420 mg at an interval of every 2 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

31. A method of reducing the frequency of atopic dermatitis exacerbation in a subject comprising administering a therapeutically effective amount of an anti-TSLP antibody or antibody variant in a dose of 280 mg to 420 mg at an interval of every 2 weeks, wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

32. The method of claim 30 or 31, wherein the antibody or antibody variant is administered every 4 weeks.

33. The method of claim 30 or 31, wherein the antibody or antibody variant is administered at a dose of 280 mg.

34. The method of claim 30 or 31, wherein the antibody or antibody variant is administered at a dose of 420 mg.

35. The method of any one of claims 30 to 34, wherein the antibody or antibody variant is administered for a period of at least 4 months, 6 months, 9 months, 1 year or more.

36. The method of any one of claims 30 to 35, wherein said anti-TSLP antibody or antibody variant is selected from the group consisting of a human antibody, a humanized antibody, a chimeric antibody, a monoclonal antibody, a recombinant antibody, an IgG1 antibody, an IgG2 antibody, an IgG3 antibody, and an IgG4 antibody.

37. The method any one of claims 30 to 36, wherein antibody or antibody variant is an IgG2 antibody.

38. The method of any one of claims 30 to 37, wherein the antibody or antibody variant is a human antibody.

39. The method of any one of claims 30 to 38, wherein the antibody or antibody variant further comprises a pharmaceutically acceptable carrier or excipient.

40. The method of any one of claims 30 to 39, wherein the administration delays the time to an atopic dermatitis exacerbation compared to a subject not receiving the anti-TSLP antibody.

41. The method any one of claims 30 to 40, wherein the administration reduces frequency of or levels of co-administered therapy in the subject.

42. The method of claim 41, wherein the co-administered therapy is selected form the group consisting of topical corticosteroids, topical calcineurin inhibitors, dupilimab, immunosuppressive or immunomodulating drugs (e.g., systemic corticosteroids, cyclosporine, mycophenolate-mofetil, interferon (IFN)-gamma, Janus kinase inhibitors, azathioprine, methotrexate), anti-IL-13 antibodies, anti-IL-5 pathway antibodies (benralizumab, mepolizumab, reslizumab), or combinations thereof.

43. The method of claim 41, wherein the administration eliminates the need for corticosteroid therapy.

44. A method for reducing Investigator's Global Assessment (IGA) and/or Eczema Area and Severity Index (EASI) score in a subject comprising administering a therapeutically effective amount of an anti-TSLP antibody or antibody variant in a dose of 280 mg to 420 mg at an interval of every 2 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

45. A method for reducing Investigator's Global Assessment (IGA) and/or Eczema Area and Severity Index (EASI) score in a subject comprising administering a therapeutically effective amount of an anti-TSLP antibody or antibody variant in a dose of 280 mg to 420 mg at an interval of every 2 weeks, wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

46. A method for reducing Investigator's Global Assessment (IGA) and/or Eczema Area and Severity Index (EASI) score in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises
a. a light chain variable domain comprising:
i. a light chain CDR1 sequence comprising the amino acid sequence set forth in SEQ ID NO:3;
ii. a light chain CDR2 sequence comprising the amino acid sequence set forth in SEQ ID NO:4;
iii. a light chain CDR3 sequence comprising the amino acid sequence set forth in SEQ ID NO:5; and
b. a heavy chain variable domain comprising:
i. a heavy chain CDR1 sequence comprising the amino acid sequence set forth in SEQ ID NO:6;
ii. a heavy chain CDR2 sequence comprising the amino acid sequence set forth in SEQ ID NO:7, and
iii. a heavy chain CDR3 sequence comprising the amino acid sequence set forth in SEQ ID NO:8, wherein the antigen binding protein specifically binds to a TSLP polypeptide as set forth in amino acids 29-159 of SEQ ID NO:2.

47. A method for reducing Investigator's Global Assessment (IGA) and/or Eczema Area and Severity Index (EASI) score in a subject comprising administering a therapeutically effective amount of an anti-TSLP antibody or antibody variant in a dose of 210 mg at an interval of every 4 weeks, wherein both binding sites of the antibody have identical binding to TSLP, and the antibody comprises
a. a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11;
iii. a sequence of amino acids encoded by a polynucleotide that hybridizes under moderately stringent conditions to the complement of a polynucleotide consisting of SEQ ID NO:11; and
b. a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9;
iii. a sequence of amino acids encoded by a polynucleotide that hybridizes under moderately stringent conditions to the complement of a polynucleotide consisting of SEQ ID NO:9; or c. a light chain variable domain of (a) and a heavy chain variable domain of (b).

48. The method of any one of claims 44 to 47 wherein the subject has an IGA score of 3, 4, or 5.

49. The method of claim any one of claims 30 to 48, wherein the anti-TSLP antibody is tezepelumab.

50. The method of claim 49, wherein the antibody is an IgG2 antibody, and has the full length heavy and light chain sequences set out in SEQ ID NOs: 105 and 106, respectively.

51. The method of any one of claims 44-50, wherein the antibody or antibody variant is administered every 4 weeks.

52. The method any one of the preceding claims, wherein the administration is subcutaneous or intravenous.


SEQUENCE LISTING (106 sequences)

SEQ ID NO: 1 - DNA, 743 bp, Homo Sapiens
  misc_feature: TSLP
  CDS (200)..(676)
gcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcccaaaataaatcatctttattaagtaaaaaaaaa

SEQ ID NO: 2 - protein, 159 aa, Homo Sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - protein, 11 aa, Homo Sapiens
  MISC_FEATURE: LCDR1
GGNNLGSKSVH

SEQ ID NO: 4 - protein, 7 aa, Homo Sapiens
  MISC_FEATURE: LCDR2
DDSDRPS

SEQ ID NO: 5 - protein, 11 aa, Homo Sapiens
  MISC_FEATURE: LCDR3
QVWDSSSDHVV

SEQ ID NO: 6 - protein, 5 aa, Homo Sapiens
  MISC_FEATURE: HCDR1
TYGMH

SEQ ID NO: 7 - protein, 17 aa, Homo Sapiens
  MISC_FEATURE: HCDR2
VIWYDGSNKHYADSVKG

SEQ ID NO: 8 - protein, 13 aa, Homo Sapiens
  MISC_FEATURE: HCDR3
APQWELVHEAFDI

SEQ ID NO: 9 - DNA, 366 bp, Homo Sapiens
  misc_feature: Heavy Chain VH
cagatgcagctggtggagtctgggggaggcgtggtccagcctgggaggtccctgagactctcctgtgcagcgtctggattcaccttcagaacctatggcatgcactgggtccgccaggctccaggcaagggactggagtgggtggcagttatatggtatgatggaagtaataaacactatgcagactccgtgaagggccgattcaccatcaccagagacaattccaagaacactctgaatctgcaaatgaacagcctgagagccgaggacacggctgtgtattactgtgcgagagcccctcagtgggagctagttcatgaagcttttgatatctggggccaagggacaatggtcaccgtctcttca

SEQ ID NO: 10 - protein, 122 aa, Homo Sapiens
  MISC_FEATURE: Heavy Chain VH
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 11 - DNA, 325 bp, Homo Sapiens
  misc_feature: Light Chain VL
tcctatgtgctgactcagccaccctcggtgtcagtggccccaggacagacggccaggattacctgtgggggaaacaaccttggaagtaaaagtgtgcactggtaccagcagaagccaggccaggcccctgtgctggtcgtctatgatgatagcgaccggccctcatggatccctgagcgattctctggctccaactctgggaacacggccaccctgaccatcagcaggggcgaagccggggatgaggccgactattactgtcaggtgtgggatagtagtagtgatcatgtggtatttcggcggagggaccaagctgaccgtccta

SEQ ID NO: 12 - protein, 108 aa, Homo Sapiens
  MISC_FEATURE: Light Chain VL
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 13 - protein, 5 aa, Artificial Sequence
  Synthetic peptide
DYWMH

SEQ ID NO: 14 - protein, 19 aa, Artificial Sequence
  Synthetic peptide
HIKSKTDAGTTDYAAPVKG

SEQ ID NO: 15 - protein, 9 aa, Artificial Sequence
  Synthetic peptide
EIYYYAFDS

SEQ ID NO: 16 - protein, 11 aa, Artificial Sequence
  Synthetic peptide
SGDNIGSKYVH

SEQ ID NO: 17 - protein, 7 aa, Artificial Sequence
  Synthetic peptide
GDNERPS

SEQ ID NO: 18 - protein, 10 aa, Artificial Sequence
  Synthetic peptide
QAADWVDFYV

SEQ ID NO: 19 - protein, 7 aa, Artificial Sequence
  Synthetic peptide
GFTFSDY

SEQ ID NO: 20 - protein, 8 aa, Artificial Sequence
  Synthetic peptide
KSKTDAGT

SEQ ID NO: 21 - protein, 7 aa, Artificial Sequence
  Synthetic peptide
DNIGSKY

SEQ ID NO: 22 - protein, 3 aa, Artificial Sequence
  Synthetic peptide
GDN

SEQ ID NO: 23 - protein, 7 aa, Artificial Sequence
  Synthetic peptide
ADWVDFY

SEQ ID NO: 24 - protein, 120 aa, Artificial Sequence
  Synthetic peptide
EVQLVESGGGLVKPGGSLRLSCAASGFTFSDYWMHWVRQAPGKGLEWVGHIKSKTDAGTTDYAAPVKGRFTISRDDSKNTLYLQMNSLKTEDTAVYYCAREIYYYAFDSWGQGTLVTVSS

SEQ ID NO: 25 - protein, 107 aa, Artificial Sequence
  Synthetic peptide
SYELTQPLSVSVALGQTARITCSGDNIGSKYVHWYQQKPGQAPVLVIYGDNERPSGIPERFSGSNSGNTATLTISRAQAGDEADYYCQAADWVDFYVFGGGTKLTVL

SEQ ID NO: 26 - protein, 223 aa, Artificial Sequence
  Synthetic peptide
EVQLVESGGGLVKPGGSLRLSCAASGFTFSDYWMHWVRQAPGKGLEWVGHIKSKTDAGTTDYAAPVKGRFTISRDDSKNTLYLQMNSLKTEDTAVYYCAREIYYYAFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 27 - protein, 213 aa, Artificial Sequence
  Synthetic peptide
SYELTQPLSVSVALGQTARITCSGDNIGSKYVHWYQQKPGQAPVLVIYGDNERPSGIPERFSGSNSGNTATLTISRAQAGDEADYYCQAADWVDFYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 28 - protein, 450 aa, Artificial Sequence
  Synthetic peptide
EVQLVESGGGLVKPGGSLRLSCAASGFTFSDYWMHWVRQAPGKGLEWVGHIKSKTDAGTTDYAAPVKGRFTISRDDSKNTLYLQMNSLKTEDTAVYYCAREIYYYAFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 213 aa, Artificial Sequence; identical to SEQ ID NO: 27
  Synthetic peptide

SEQ ID NO: 30 - protein, 149 aa, Artificial Sequence
  Synthetic peptide
MGSSHHHHHHLEVLFQGPYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 31 - protein, 107 aa, Artificial
  Dom30h-440-81/86 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCVQIGEDPVTFGQGTKVEIK

SEQ ID NO: 32 - protein, 11 aa, Artificial
  CDRL1 of Dom30h-440-81/86, Dom30h-440-53, Dom30h-440-54, Dom30h-440-55, Dom30h-440-56, Dom30h-440-57, Dom30h-440-58, Dom30h-440-60, Dom30h-440-63, Dom30h-440-64 and Dom30h-440-65 (Kabat, Chothia and AbM CDR definition)
RASRPIRNWLD

SEQ ID NO: 33 - protein, 7 aa, Artificial
  CDRL2 of Dom30h-440-81/86, Dom30h-440-53, Dom30h-440-54, Dom30h-440-55, Dom30h-440-56, Dom30h-440-57, Dom30h-440-58, Dom30h-440-60, Dom30h-440-63, Dom30h-440-64 and Dom30h-440-65 (Kabat, Chothia, AbM CDR definition)
GASHLQS

SEQ ID NO: 34 - protein, 9 aa, Artificial
  CDRL3 of Dom30h-440-81/86 and Dom30h-440-55 (Kabat, Chothia, AbM CDR definition)
VQIGEDPVT

SEQ ID NO: 35 - protein, 10 aa, Artificial
  CDRL2 of Dom30h-440-81/86 (Contact CDR definition)
LLIWGASHLQ

SEQ ID NO: 36 - protein, 5 aa, Mus musculus
RYNVH

SEQ ID NO: 37 - protein, 16 aa, Mus musculus
MIWDGGSTDYNSALKS

SEQ ID NO: 38 - protein, 6 aa, Mus musculus
NRYESG

SEQ ID NO: 39 - protein, 17 aa, Mus musculus
KSSQSLLNSGNRKNYLT

SEQ ID NO: 40 - protein, 7 aa, Mus musculus
WASTRES

SEQ ID NO: 41 - protein, 12 aa, Mus musculus
QNDYTYPFTFGS

SEQ ID NO: 42 - protein, 5 aa, Mus musculus
AYWMS

SEQ ID NO: 43 - protein, 17 aa, Mus musculus
EINPDSSTINCTPSLKD

SEQ ID NO: 44 - protein, 11 aa, Mus musculus
RLRPFWYFDVW

SEQ ID NO: 45 - protein, 16 aa, Mus musculus
RSSQSIVQSNGNTYLE

SEQ ID NO: 46 - protein, 7 aa, Mus musculus
KVSNRFS

SEQ ID NO: 47 - protein, 9 aa, Mus musculus
FQGSHVPRT

SEQ ID NO: 48 - protein, 6 aa, Mus musculus
TDYAWN

SEQ ID NO: 49 - protein, 16 aa, Mus musculus
YIFYSGSTTYTPSLKS

SEQ ID NO: 50 - protein, 8 aa, Mus musculus
GGYDVNYF

SEQ ID NO: 51 - protein, 11 aa, Mus musculus
LASQTIGAWLA

SEQ ID NO: 52 - protein, 7 aa, Mus musculus
AATRLAD

SEQ ID NO: 53 - protein, 9 aa, Mus musculus
QQFFSTPWT

SEQ ID NO: 54 - protein, 5 aa, Mus musculus
GYTMN

SEQ ID NO: 55 - protein, 16 aa, Mus musculus
LINPYNGVTSYNQKFK

SEQ ID NO: 56 - protein, 8 aa, Mus musculus
GDGNYWYF

SEQ ID NO: 57 - protein, 11 aa, Mus musculus
SASSSVTYMHW

SEQ ID NO: 58 - protein, 7 aa, Mus musculus
EISKLAS

SEQ ID NO: 59 - protein, 9 aa, Mus musculus
QEWNYPYTF

SEQ ID NO: 60 - protein, 117 aa, Mus musculus
QVQLQESGPGLVAPSQSLSITCTVSGFSLSRYNVHWVRQPPGKGLEWLGMIWDGGSTDYNSALKSRLSITKDNSKSQVFLKMNSLQTDDTAMYYCARNRYESGMDYWGQGTTVTVSS

SEQ ID NO: 61 - protein, 114 aa, Mus musculus
DIVMTQTPSSLTVTAGEKVTMSCKSSQSLLNSGNRKNYLTWYQQKPGQSPKLLIYWASTRESGVPDRFTGSGSGTDFTLIISSVQAEDLAVYYCQNDYTYPFTFGSGTKLEIKR

SEQ ID NO: 62 - protein, 119 aa, Mus musculus
EVKLLESGGGLVQPGGSLKLSCAASGFAFSAYWMSWVRQAPGKGLEWIGEINPDSSTINCTPSLKDKFIISRDNAKNTLSLQMNKVRSEDTALYYCARRLRPFWYFDVWGAGTTVTVSS

SEQ ID NO: 63 - protein, 112 aa, Mus musculus
DVLMTQTPLSLPVSLGDQASISCRSSQSIVQSNGNTYLEWYLQKPGQSPKLLIYKVSNRFSGVPDRFSGSGSGTDFTLKISRVEAEDLGVYYCFQGSHVPRTFGGGTKLEIK

SEQ ID NO: 64 - protein, 119 aa, Mus musculus
DVQLQESGPGLVKPSQSLSLTCTVTGYSITTDYAWNWIRQFPGNKLEWMGYIFYSGSTTYTPSLKSRISITRDTSKNQFFLQLNSVTTEDTATYYCARGGYDVNYFDYWGQGTTLTVSS

SEQ ID NO: 65 - protein, 107 aa, Mus musculus
DIQMTQSPASQSASLGESVTITCLASQTIGAWLAWYQQKPGKSPQLLIYAATRLADGVPSRFSGSGSGTKFSFKISSLQAEDFVSYYCQQFFSTPWTFGGGTKLEIK

SEQ ID NO: 66 - protein, 119 aa, Mus musculus
EVQLQQSGPELLKPGASMKISCKASGYSFTGYTMNWVKQSHGKNLEWIGLINPYNGVTSYNQKFKGKATLTVAKSSSTAYMELLSLTSEDSAVYYCARGDGNYWYFDVWGAGTTVTVSS

SEQ ID NO: 67 - protein, 105 aa, Mus musculus
EIVLTQSPAITAASLGQKVTITCSASSSVTYMHWYQQKSGTSPKPWIYEISKLASGVPVRFSGSGSGTSYSLTISSMEAEDAAIYYCQEWNYPYTFGGGTKLEIK

SEQ ID NO: 68 - protein, 119 aa, Mus musculus
EVQLQQSGPELVKPGASMKISCKASGYSFTGYTMNWVKQSHGKNLEWIGLINPYSGITSYNQNFKGKATLTVDKSSSTAYMELLNLTSEDSAVYYCARGDGNYWYFDVWGAGTTVTVSS

SEQ ID NO: 69 - protein, 105 aa, Mus musculus
EIILTQSPAITAASLGQKVTITCSASSSVSYMHWYQQKSGTSPKPWIYEISKLASGVPARFSGSGSGTSYSLTISSMEAEDAAIYYCQYWNYPYTFGGGTKLEIK

SEQ ID NO: 70 - protein, 5 aa, Mus musculus
GYTMN

SEQ ID NO: 71 - protein, 16 aa, Mus musculus
LINPYSGITSYNQNFK

SEQ ID NO: 72 - protein, 8 aa, Mus musculus
GDGNYWYF

SEQ ID NO: 73 - protein, 11 aa, Mus musculus
SASSSVSYMHW

SEQ ID NO: 74 - protein, 7 aa, Mus musculus
EISKLAS

SEQ ID NO: 75 - protein, 9 aa, Mus musculus
QYWNYPYTF

SEQ ID NO: 76 - protein, 12 aa, Cricetulus migratorius
GFSITTSGYYWT

SEQ ID NO: 77 - protein, 16 aa, Cricetulus migratorius
YIGYNSKTYYNPSLKS

SEQ ID NO: 78 - protein, 12 aa, Cricetulus migratorius
SLYGGYKDAFDS

SEQ ID NO: 79 - protein, 11 aa, Cricetulus migratorius
KASQSIGTSLH

SEQ ID NO: 80 - protein, 7 aa, Cricetulus migratorius
FASRSIT

SEQ ID NO: 81 - protein, 9 aa, Cricetulus migratorius
QQSPGFPPT

SEQ ID NO: 82 - protein, 122 aa, Cricetulus migratorius
QIQLQESGPGLVKPSQSLSLTCSVTGFSITTSGYYWTWIRQFPGKKLEWMGYIGYNSKTYYNPSLKSRISITRDTSKNQFLLHLNSVTTEDTATYYCARSLYGGYKDAFDSWGQGTLVTVSS

SEQ ID NO: 83 - protein, 108 aa, Cricetulus migratorius
DVVLTQTPATLSAIPGERVTMTCKASQSIGTSLHWYQHRPNETPRLLIKFASRSITGIPSRFSGSGSGTDFTLGINNLEAEDFAIYYCQQSPGFPPTFGSGTKLEINR

SEQ ID NO: 84 - protein, 12 aa, Artificial
  hCDR1 TSLPR-012_166
GFSITTSGYYWS

SEQ ID NO: 85 - protein, 16 aa, Artificial
  hCDR2 TSLPR-012_141
YIGYNSKTYYNPALKS

SEQ ID NO: 86 - protein, 11 aa, Artificial
  lCDR1 TSLPR-012_141
RASQSIGTSLH

SEQ ID NO: 87 - protein, 7 aa, Artificial
  lCDR2 TSLPR-012_141
FASRLQS

SEQ ID NO: 88 - protein, 7 aa, Artificial
  lCDR2 TSLPR-012_75
FASRSIS

SEQ ID NO: 89 - protein, 122 aa, Artificial
  sequence TSLPR-012_141 (humanized) HC variable region
QVQLVESGGGLVKPGGSLRLSCAASGFSITTSGYYWTWIRQFPGKGLEWMGYIGYNSKTYYNPALKSRITISRDNAKNSLYLQMNSLRAEDTAVYYCARSLYGGYKDAFDSWGQGTLVTVSS

SEQ ID NO: 90 - protein, 108 aa, Artificial
  sequence TSLPR-012_141 (humanized) LC variable region
DIQMTQSPSSLSASVGDRVTITCRASQSIGTSLHWYQQKPGKAPKLLIKFASRLQSGVPSRFSGSGSGTDFTLTISSLQPEDFATYYCQQSPGFPPTFGQGTKLEIKR

SEQ ID NO: 91 - protein, 108 aa, Artificial
  sequence TSLPR-012_75 (humanized) LC variable region
EIVLTQSPDFQSVTPKEKVTITCRASQSIGTSLHWYQQKPDQSPKLLIKFASRSISGVPSRFSGSGSGTDFTLTINSLEAEDAATYYCQQSPGFPPTFGQGTKLEIKR

SEQ ID NO: 92 - protein, 122 aa, Artificial
  sequence TSLPR-012_166 (humanized) HC variable region
QVQLQESGPGLVKPSQTLSLTCTVSGFSITTSGYYWSWIRQHPGKGLEWIGYIGYNSKTYYSPSLKSRVTISRDTSKNQFSLKLSSVTAADTAVYYCARSLYGGYKDAFDSWGQGTLVTVSS

SEQ ID NO: 93 - protein, 108 aa, Artificial
  sequence TSLPR-012_166 (humanized) LC variable region
DIQMTQSPSSLSASVGDRVTITCRASQSIGTSLHWYQHRPGETPKLLIKFASRSISGVPSRFSGSGSGTDFTLTISSLQPEDFATYYCQQSPGFPPTFGQGTKLEIKR

SEQ ID NO: 94 - protein, 108 aa, Artificial
  sequence TSLPR-012_189 (humanized) LC variable region
EIVMTQSPATLSVSPGERATLSCRASQSIGTSLHWYQQKPGQAPRLLIKFASRSITGIPARFSGSGSGTEFTLTISSLQSEDFAVYYCQQSPGFPPTFGQGTKLEIKR

SEQ ID NO: 95 - protein, 10 aa, Artificial Sequence
  Synthetic peptide
GYIFTDYAMH

SEQ ID NO: 96 - protein, 17 aa, Artificial Sequence
  Synthetic peptide
TFIPLLDTSDYAQKFQG

SEQ ID NO: 97 - protein, 11 aa, Artificial Sequence
  Synthetic peptide
MGVTHSYVMDA

SEQ ID NO: 98 - protein, 11 aa, Artificial Sequence
  Synthetic peptide
RASQPISISVH

SEQ ID NO: 99 - protein, 7 aa, Artificial Sequence
  Synthetic peptide
FASQSIS

SEQ ID NO: 100 - protein, 9 aa, Artificial Sequence
  Synthetic peptide
QQTFSLPYT

SEQ ID NO: 101 - protein, 120 aa, Artificial Sequence
  Synthetic peptide
QVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSS

SEQ ID NO: 102 - protein, 109 aa, Artificial Sequence
  Synthetic peptide
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIKRT

SEQ ID NO: 103 - protein, 450 aa, Artificial Sequence
  Synthetic peptide
QVQLVQSGAEVKKPGASVKVSCKASGYIFTDYAMHWVRQAPGQGLEWMGTFIPLLDTSDYAQKFQGRVTMTADTSTSTAYMELRSLRSDDTAVYYCARMGVTHSYVMDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 214 aa, Artificial Sequence
  Synthetic peptide
EIVLTQSPGTLSLSPGERATLSCRASQPISISVHWYQQKPGQAPRLLIYFASQSISGIPDRFSGSGSGTDFTLTISRLEPEDFAVYYCQQTFSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 448 aa, Artificial Sequence
  Synthetic Polypeptide
  MISC_FEATURE: Heavy chain
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 106 - protein, 214 aa, Artificial Sequence
  Synthetic Polypeptide
  MISC_FEATURE: Light Chain
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS